(19) United States
(12) Patent Application Publication
(10) Pub. No.: US 2024/0070627 A1
(43) Pub. Date: Feb. 29, 2024

(54) Healthcare Blockchain Cryptosystem

(71) Applicant: Louis Galterio, North Port, FL (US)

(72) Inventor: Louis Galterio, North Port, FL (US)

(21) Appl. No.: 18/452,594

(22) Filed: Aug. 21, 2023

Related U.S. Application Data
(60) Provisional application No. 63/373,099, filed on Aug. 22, 2022.

(51) Int. Cl. G06Q 20/06; G06Q 20/02; G06Q 20/36; G06Q 50/22

(52) U.S. Cl. CPC G06Q 20/065; G06Q 20/02; G06Q 20/36; G06Q 50/22; G06Q 2220/00

(57) ABSTRACT

Technologies discussed herein secure access to a Healthcare Blockchain. This apparatus is a collection of hardware and software. It utilizes new to the art encoded firmware specific to a patient or entities' unique identity as collection antennae to bridge to Web3 Blockchain distributed networks. It solves the problems in healthcare technology of incorrect, inconsistent, non-interoperable and disorganized data and patient information. Data normalization and validation, and continued processing occur via two way Oracles. Enablement of best embodiment for a sample use case is included in the Detailed Description and consists of A) Our “Internet of Things” or “IoT” antenna's coupled with software and B) New integrated software built into emergent blockchain tokens, API's, and smart contracts (simultaneously existing as firmware based “Smart Contracts” to pair with software counterparts). These new apparatus components along with various methods, creates our Web3 based Blockchain with integration pathway to Web2 and Web1 applications.

### TECHNICAL FIELD

The invention is related to the technical field of Blockchain and Web 3 distributed databases. Blockchain is a distributed electronic ledger running across a world-wide system of internet nodes and functions similar to internet distributed name services.

### BACKGROUND THE INVENTION

From a technical perspective, the field of this invention generally relates to blockchain ledger entries (or resources) supported and validated based on consensus protocols. It is focused on the health care technology ecosystem. Due to the generational variation in the use of technology in healthcare, there exist multiple instances of incompatible software systems and incompatible approaches to the format of a standard record designation for patients and entities. There is need for a normalization mechanism that can solve this issue while taking into account the nature of constant change in the industry.

Various web resources may already exists in the public domain, either in the form of publication or usage and act as sources of information or motivation to the inception of the invention. Approaches over time in Web 1 and Web 2, and even from classical programming, were established to address specific problems or within specific software products for unique customers. As healthcare has evolved the need for interoperability of these systems is critical from cost, utilization, and patient safety perspectives.

### BRIEF SUMMARY OF THE INVENTION

This introduces concepts further described in the Detailed Description. It is not intended to limit the scope of the claims.

The Healthcare Blockchain Crypto system encompasses integrated mechanisms and bridges software applications. The purpose is to correct health data that is inconsistent, wrong, unsecured, and non-interoperable with multiple technologies.

One object of the invention is to normalize and improve software and hardware incompatibilities within a dynamically changing environment. Another object of the invention is to insure data validation is consensus driven and interoperable across all systems affected so as to solve existing problems. Another object of the invention is to tokenize revenue flow through healthcare software and allocate payments and rewards to patients, payers, providers, and pharmacies in a system where graphene based antenna SIM cards guarantee identity of the actors.

In some embodiments, the present invention comprises telephony s, in other embodiments the present invention hyperlinks to tokenized smart contracts.

### BRIEF DESCRIPTION DRAWINGS

FIG. 1: This figure relates to claims 1-8 and lists in table format, the technology innovations created by and within the Healthcare Blockchain Cryptosystem. The apparatus and system is composed of hardware, software, and methods.

FIG. 2: This figure relates to claim 8. It is a pictorial representation of the Medical Hub Exchange described in the detailed description.

FIG. 3: Cross Walked Mapping Entity Systems (Five Lists Notated A-E). This figure relates to claim 7 and illustrates the abundance of key databases and lists in health care that must be treated as joined parameters by providers.

FIG. 4: This figure relates to both claims 1 and 8. It shows a high level interactions overview of bi-directional bridging between Physical and Cryptosystem like Metaverse (Mirror Database) and addresses spontaneous smart contract evolution based on natural selection and neuro network programming and networks.

FIG. 5: This figure presents a pictorial overview of existing Iot (Internet of Things) technology. This figure relates to both claims 1 and 2.

FIG. 6: This figure illustrates the “Handshaking Co-Processes”. When interactions occur between the hardware and embedded firmware, this handshaking “co-process” occurs and relates to claim 2 and by extension, to claim 1 as well.

FIG. 7: This figure illustrates a high level “Process Schematic Flow Overview.” This is a conceptual process diagram and relates to claim 8 and, by extension, claims 1 and 2.

FIG. 8: This figure illustrates the “Opportunistic Blockchain Ecosystem.” It illustrates that there are existing tokens, technologies, and other blockchain networks (both in existence and in a conceptual stage) that the healthcare blockchain cryptosystem interfaces with through unique oracle hardware and software bridges and is related to claim 8.

FIG. 9: This figure illustrates token classes and positions RHIOCOIN as well as summarizes the creation process of new and emergent tokens derivative to the native RHIOTOKEN. This figure relates to claims 4 and 5.

FIG. 10: This overall flowchart process presented as 3 sub-process flowcharts is named “Patient Flowcharts” and relates to claim 5 and by extension, to claim 6. It presents an initial use case example of the invention related to payments via Claims Processing; Revenue; and Real-time Quality Score Updating on a Blockchain Ledger with details of the steps found in the following DETAILED DESCRIPTION OF THE DRAWINGS section.

FIG. 11: This figure is a pictorial overview of the Healthcare Designated Record Set as proposed in draft discussion format by the Sequoia Project, October, 2022.

FIG. 12: This figure is a representative example that illustrates text queries from me and responses from an AI program, ChatGBT. These were created following a request by me to provide required steps necessary to create schematics appropriate for submitting to a material or chip card engineer for development of our physical graphene based antenna. A second example query follows on taking that output and using it to build Solidity code in the blockchain.

### DETAILED DESCRIPTION OF THE DRAWINGS

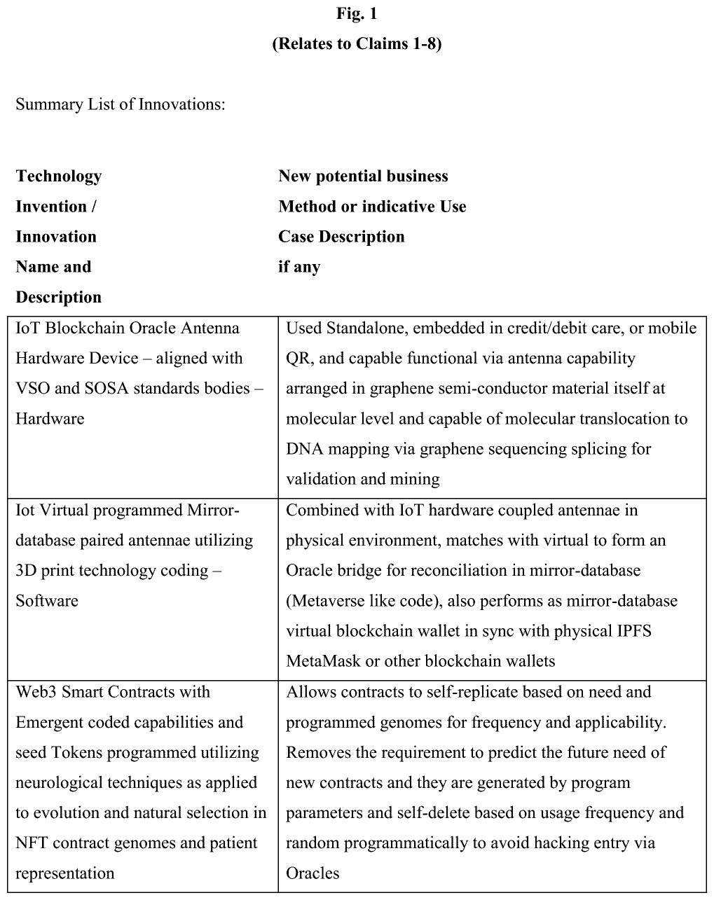

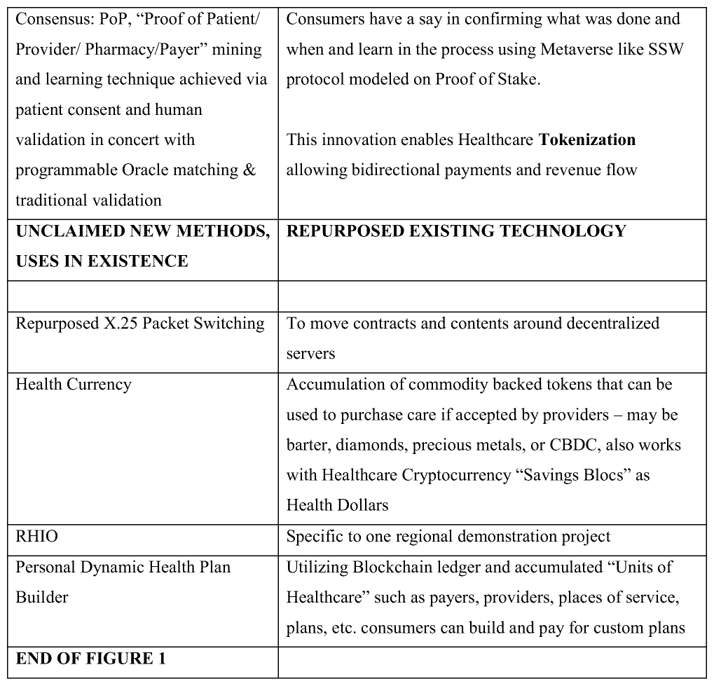

FIG. 1: This figure relates to claims 1-8 and lists in table format, the technology innovations created by and within the Healthcare Blockchain Cryptosystem. The apparatus and system is composed of hardware, software, and methods. Concurrent and newly evolved innovations, using coincident sample use cases that naturally potentiate additional innovations are further described in the detailed description under the enablement section of “How to Make and Use” the invention.

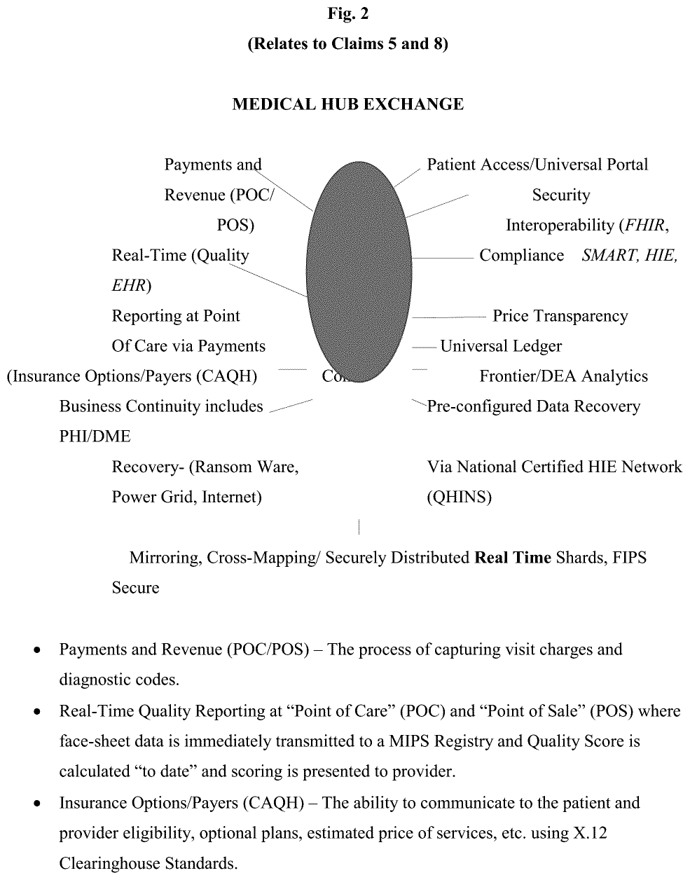

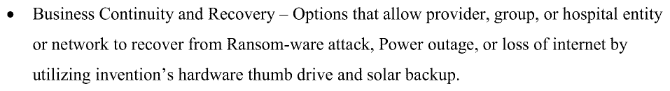

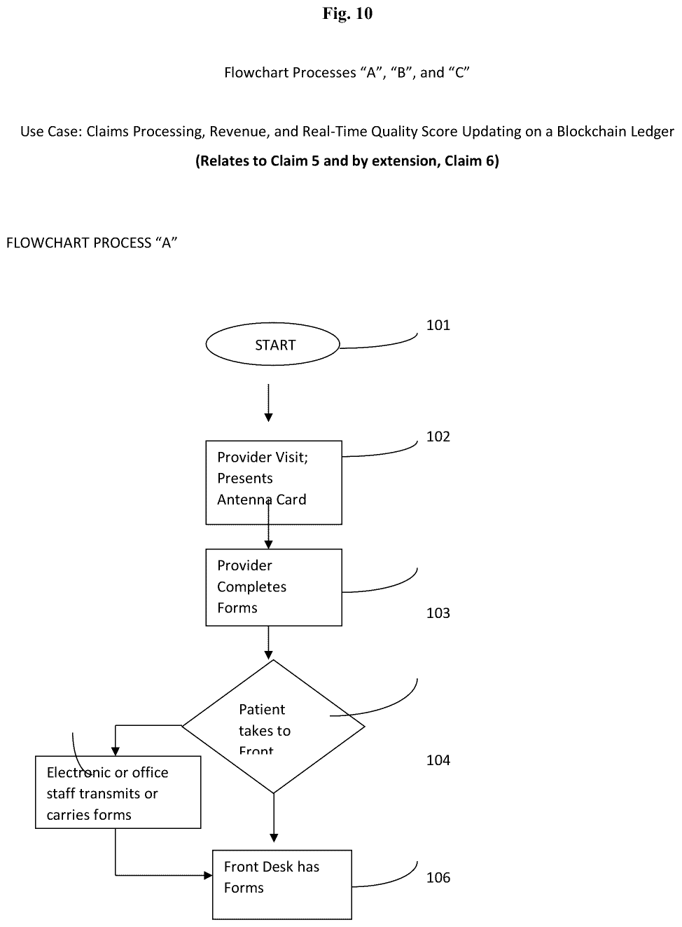

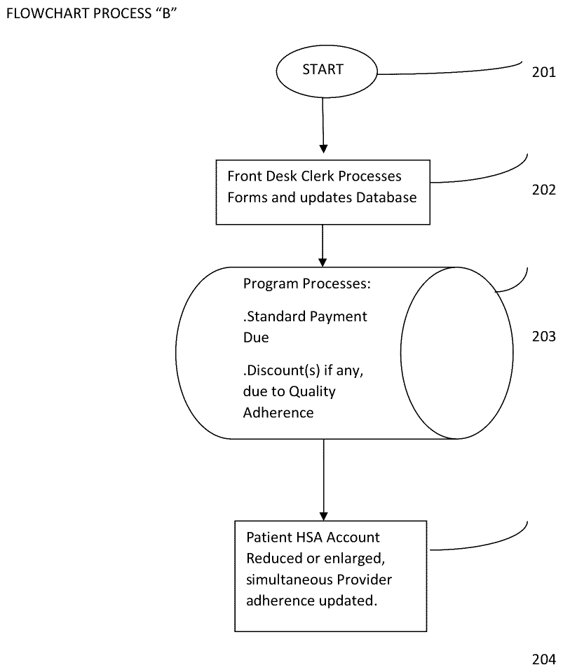

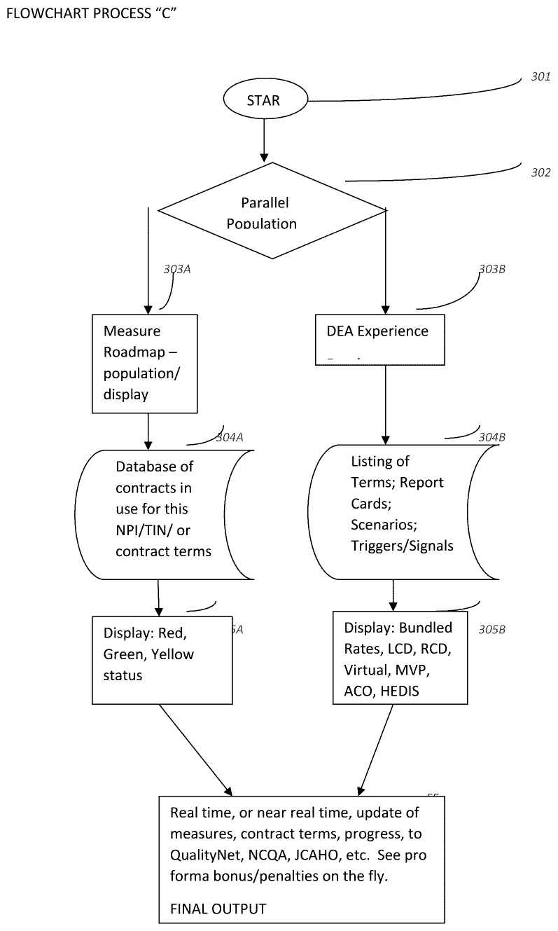

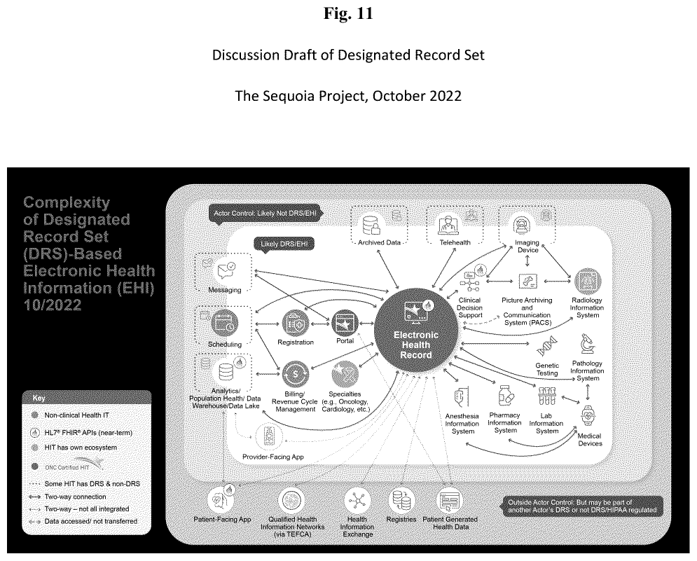

FIG. 2: This figure relates to claims 5 and 8. It is a pictorial representation of the Medical Hub Exchange described in the detailed description. It represents the following:
Payments and Revenue (POC/POS). This refers to the process of capturing visit charges and diagnostic codes.
Real-Time Quality Reporting at “Point of Care” (POC) and “Point of Sale” (POS) where face-sheet data is immediately transmitted to a MIPS Registry and Quality Score is calculated “to date” and scoring is presented to provider. (This is described in detail in FIG. 10 Flowchart and is the last Figure for the Invention).
Insurance Options/Payers (CAQH)—The ability to communicate to the patient and provider eligibility, optional plans, estimated price of services, etc. using X.12 Clearinghouse Standards.
Business Continuity and Recovery—Options that allow provider, group, or hospital entity or network to recover from Ransom-ware attack, Power outage, or loss of internet by utilizing invention's hardware thumb drive and solar backup.

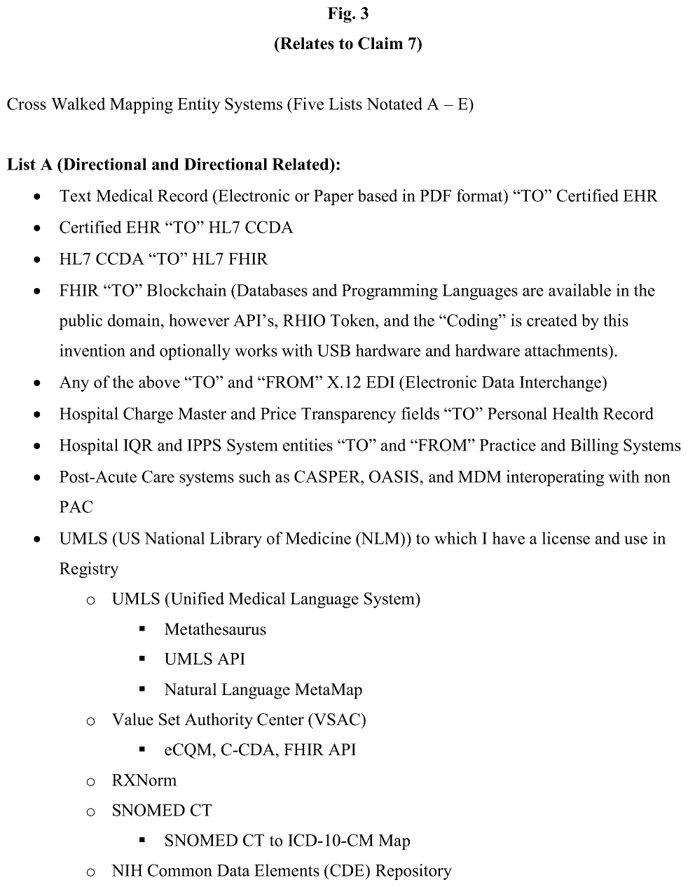

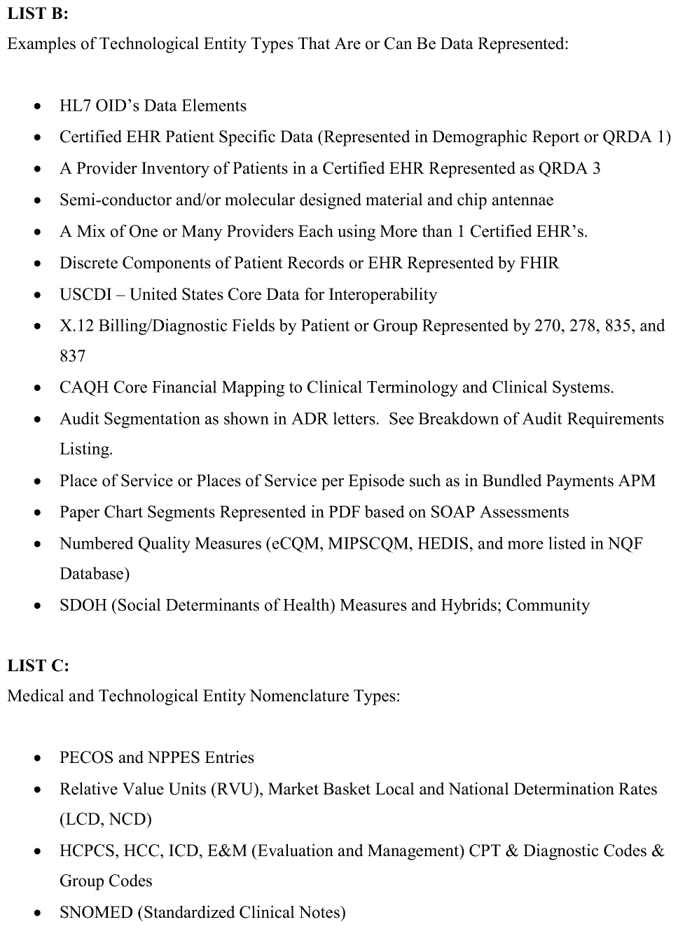

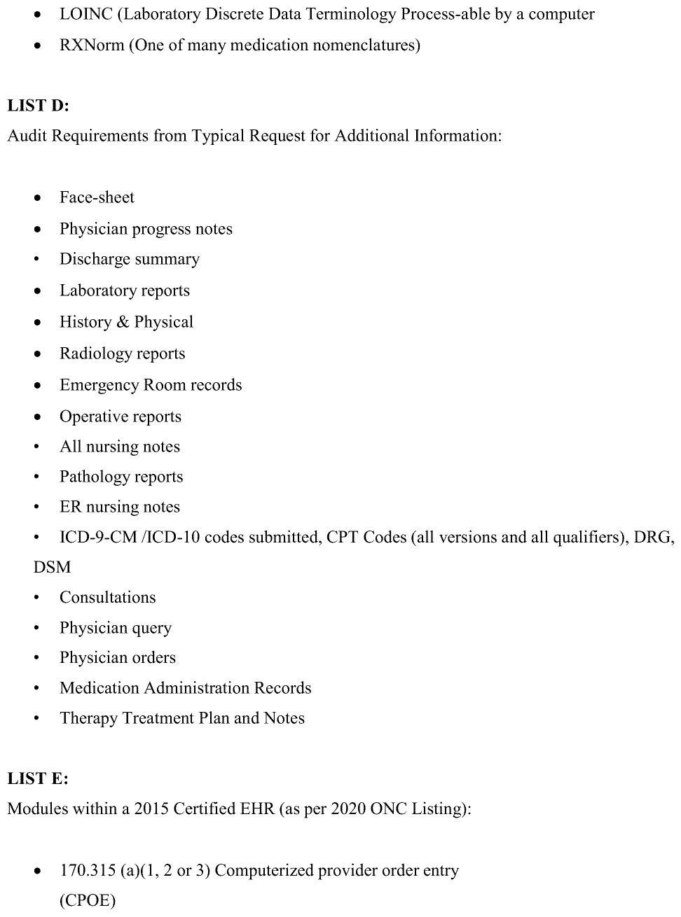

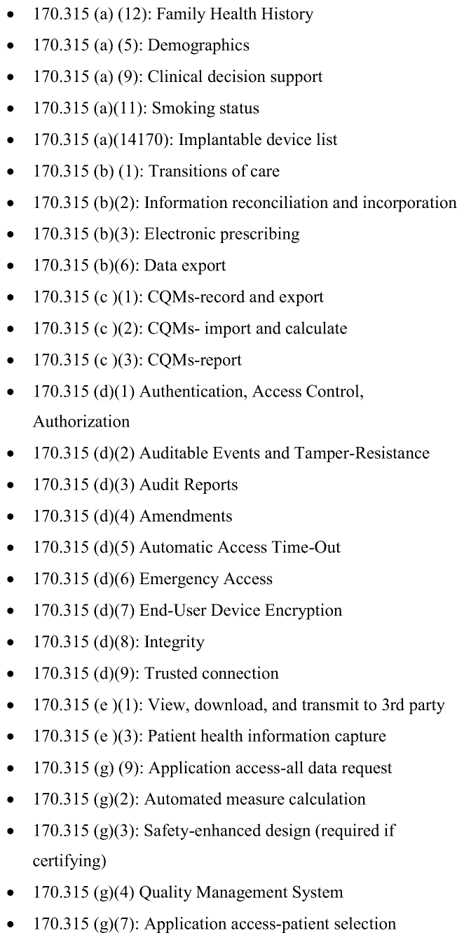

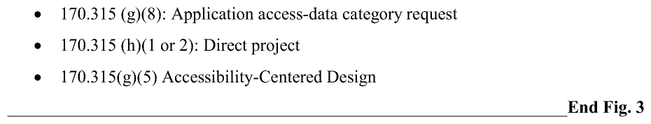

FIG. 3: These lists describe various nomenclature that, at times, utilize various words or terms to refer to the same condition, symptom, disease cluster, code classification, electronic health record and Health Information Exchange common fields and many others.

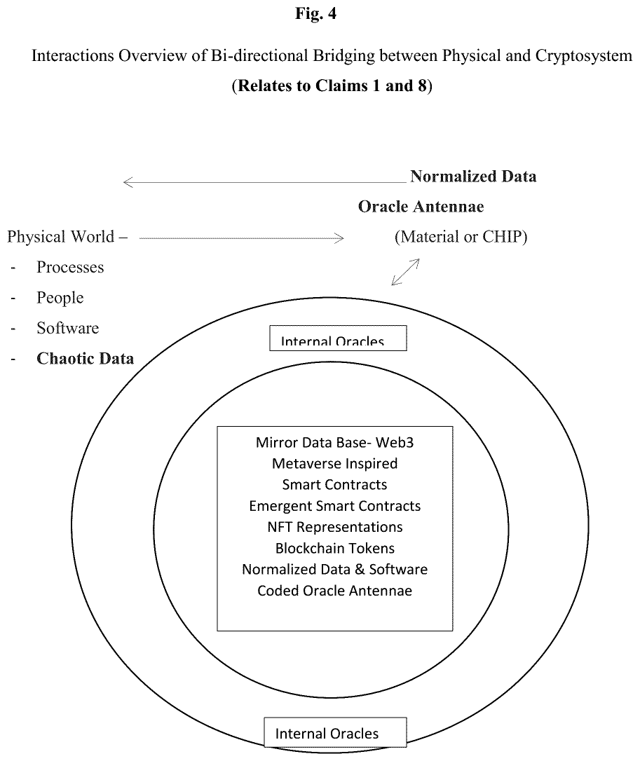

FIGS. 4-7: These Drawings are sufficiently described in the BRIEF DESCRIPTION OF DRAWINGS and no further detail is required.

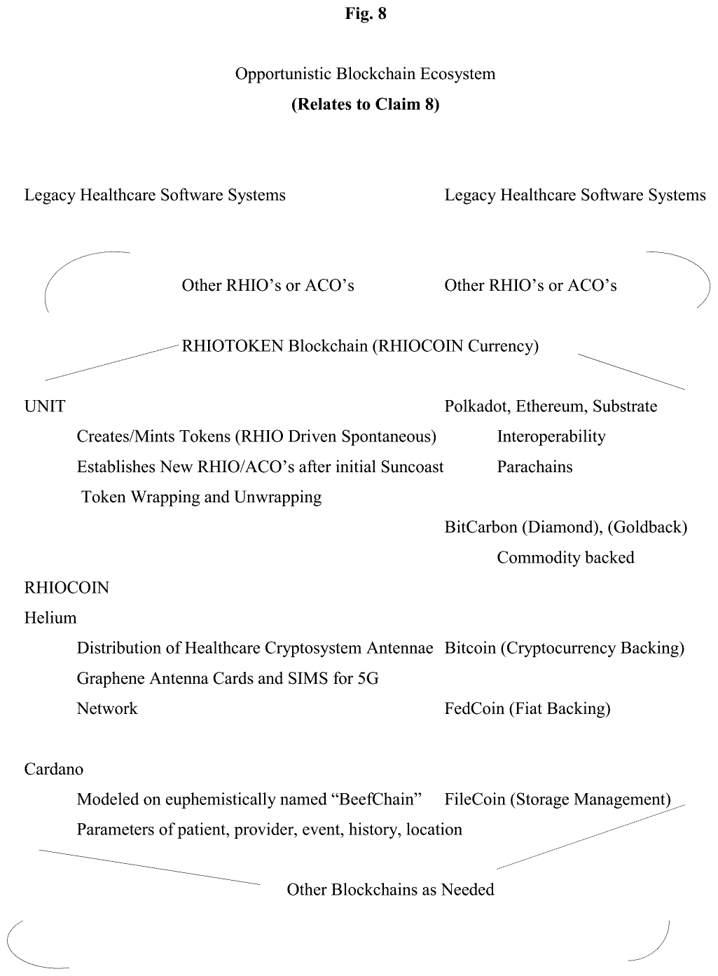

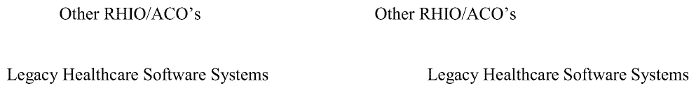

FIG. 8: Additional note, our architecture allows the creation of new smart contracts based on evolutionary parameters of natural selection via neuro programming.

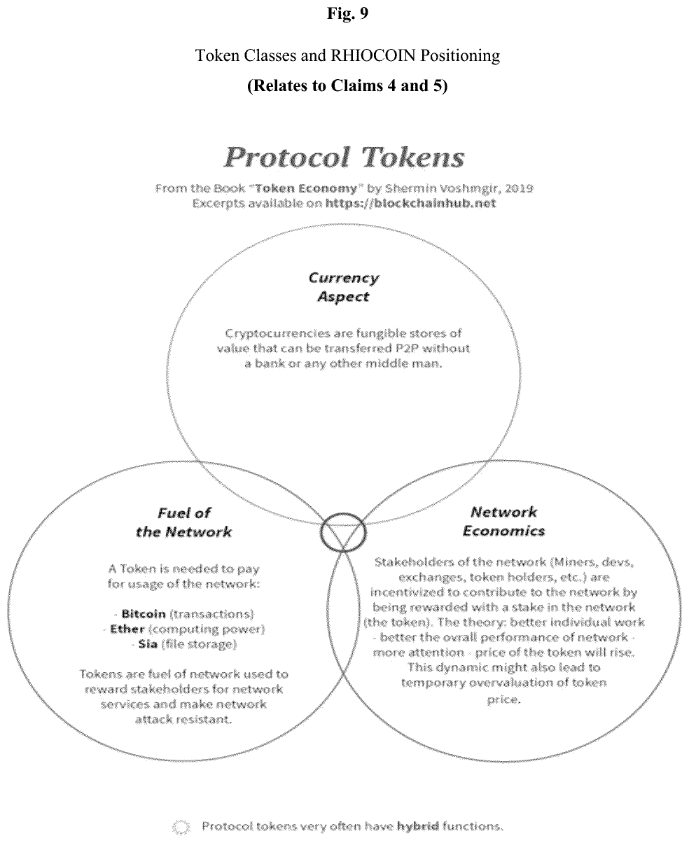

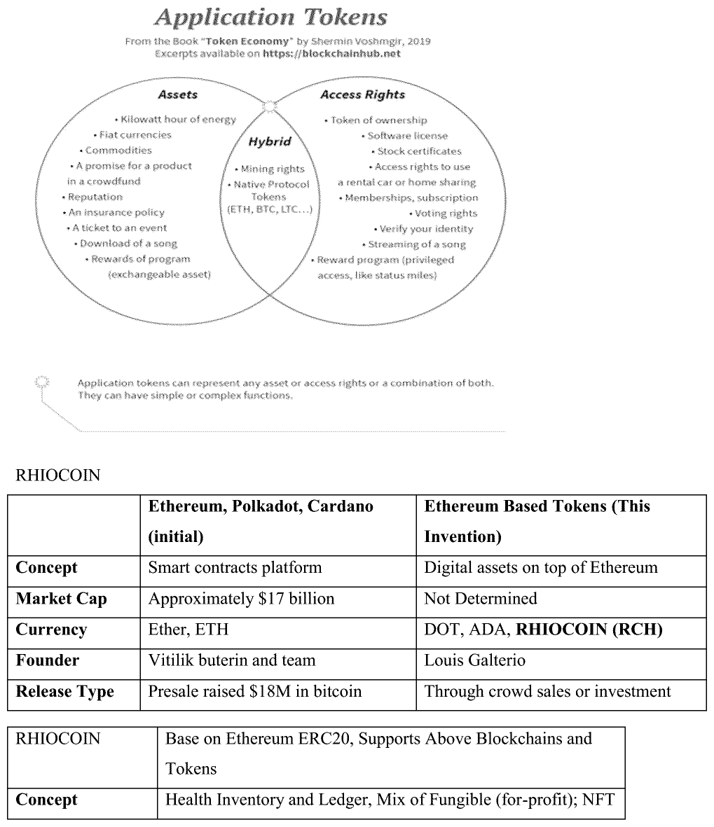

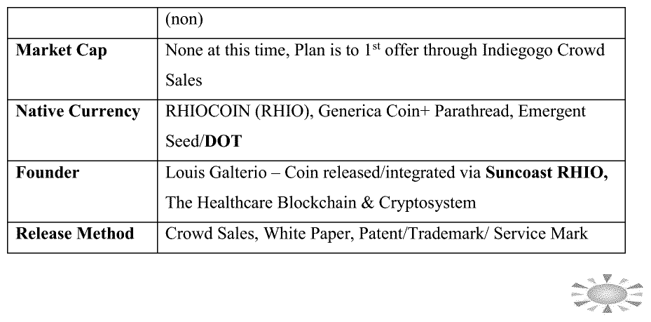

FIG. 9: These Drawings are sufficiently described in the BRIEF DESCRIPTION OF DRAWINGS and no further detail is required.

FIG. 10: As stated in the BRIEF DESCRIPTION OF THE DRAWINGS section above, this flowchart drawing is broken into 3 sub processes each with their own flowchart: Flowchart “A” addresses Pre-process patient/provider activities; Flowchart “B” addresses Payment/quality measure updating; and Flowchart “C” addresses Post Process Activities using DEA (Data Envelopment Analysis) as a Quality Measure Roadmap with real time populating of provider contracts appending provider experience to their own performance Record keeping.

Processes
A) Pre-process patient/provider activities—FLOWCHART PROCESS “A”
B) Payment/quality measure update process—FLOWCHART PROCESS B
C) Post Process—DEA Measure Roadmap/contract experience populate—FLOWCHART PROCESS C

Flowchart A) Pre-Process Patient/Provider Activities STEPS as Matched to Drawings
Patient completes doctor or provider encounter (101, 102)
Physician or provider that renders care and has a national provider identify number (NPI), reviews/updates/creates Face-sheet and superbill for patient to bring copy to front desk (or front desk receives these documents from a source other than the patient—electronic or medical office personnel) (103)
   Contains:
      Names of procedures done
      Requirement for other tests if needed
      Any referral information
      Should have diagnosis, procedure, and encounter codes if possible
Patient advances to payment window; starts Payment/metric update process (104-106)

Flowchart B) Payment/Quality Measure Update Process STEPS as Matched to Drawings

Patient presents payment to medical office check-out staff with required documents and antenna card or barcode from mobile device. (201)

Clerk performs data entry for bill settlement to cover payment and to update physician(s), other medical provider(s) (if any) for Face-sheet and super bill measure updates. (202)

Measures populate either directly from superbill and codes or is entered into local program for batch upload to update measures later. (203)

At this point, program processes: (204)
Health Savings Account (HSA) Processing
Standard payment processing
Discount(s) if any, due to quality measure target attainment or adherence
System displays or prints measure dashboard showing progress and eligible patients for goal
Shows eligible patients, measures, attribution information for episode
Sends to appropriate cryptographic medium such as Blockchain via Oracle
Patient has account reduced or pays standard rate as displayed by price list determined by combination of fee's required and offset by patient adherence and payer recommendations.
Patient can see activity since last visit, increased healthy profile as determined by plan/doctor

Flowchart C) Post Process—DEA Measure Roadmap/Contract Population STEPS as Matched to Drawings

Parallel populate/Data Envelopment Analysis (DEA) driver with visual or chart output (302)
Parallel populate—Path A (303A) or Path B (303B)

Path A—Measure Roadmap—Mapping software version or charted—provider preference (304A)
Based on contract milestones illustrated as color-coded topography: (305A)
   RED—Due
   GREEN—Attained
   YELLOW—Optional and potentially significant to clinical care; revenue optimization; contract term achievement; or any combination; can be dynamically set.

Path B—DEA Experience Database population for future contract formation or evidence (304B)
Accumulation of experience (305B)
Determine ongoing and new best practices (305B)
Assign to Tax Id that bills (TIN) in combination with NPI's that are rendering or by Group.
Lists terms
Creates Report Cards
Display varying scenarios and goals with patient attribution classification; account for variations if patient changes demographics during reporting period
Bundled episode rates
Can be local or virtual

Bi-Directional External Samples:
Quality Measure Data Bank—fed by National Quality Forum (NQF)
Rural Healthnet Consortia—vendor neutral archive
Payer Agreement Contractual Database
QPP, PARS, eCQM and AHRQ government databases
NCQA, HEDIS
IQR and OQR
QECP Data
QCDR shared measures
API Library
837 files and claims
Virtual Group entities to account for LCD and RCD—CAQH CORE
JCAHO, Leapfrog, Other

Endpoint Processing Output for Flowchart Process “C”—Forwards to the Suncoast RHIO consortium based repository. This repository contains tools for standardization of key patient data needed for quality measurement and assists in fostering ongoing standardization of data for measures. It has other development tools such as libraries of data elements that allow new electronic measures to be captured, calculated, and reported for use by clinicians and consumers.

This supports alignment of measures across 3^{rd} parties for access and reporting needs using API libraries (Application Programming Interfaces)—code that allows unlike computer software to communicate and pass parameters to each other and do not lose meaning in transition; provides for aggregating and linking “Value Based Data” for payments and care, data transparency, data consolidation, and alignment across payers.

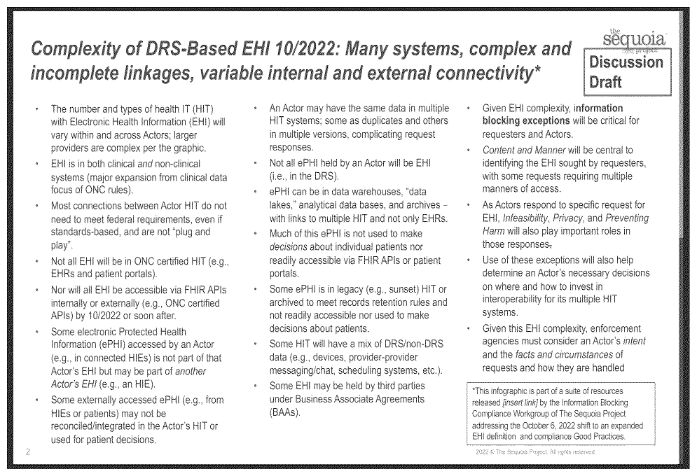

FIG. 11: Pictorial representation representing the Healthcare Designated Record Set. This figure relates to claim 9 and illustrates fields that would be weighted in the blockchain ledger file based standard designated record set. Similar to FIG. 3, this standardized weighted file incorporates lists that describe various nomenclature that, utilize various words or terms to refer to the same condition, symptom, disease cluster, code classification, electronic health record and Health Information Exchange common fields, the patient record, and many others.

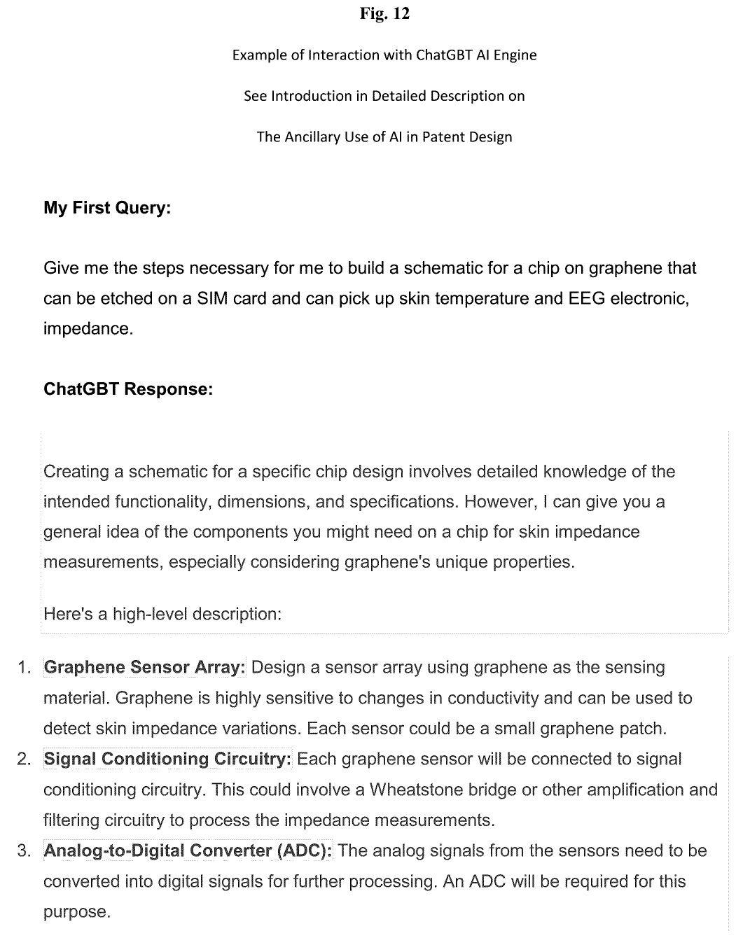

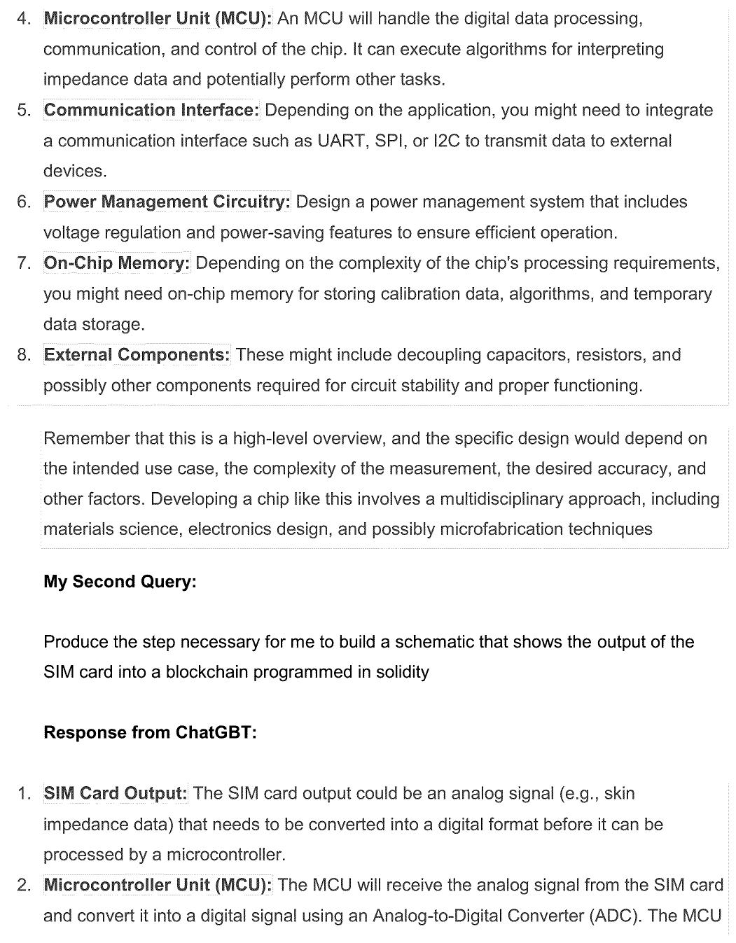

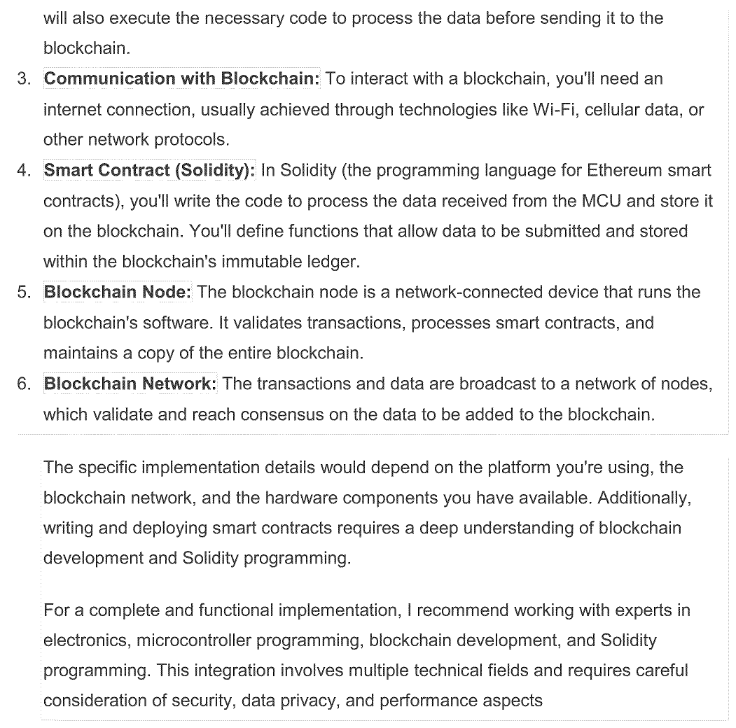

FIG. 12: These Drawings are sufficiently described in the BRIEF DESCRIPTION OF DRAWINGS and no further detail is required.

### BEST MODE FOR CARRYING OUT THE INVENTION

This topic is fully covered in the Detailed Description. In short, in some embodiments, the apparatus of the present Healthcare Cryptosystem invention comprises linked and encoded SIM and eSIM cards in 5G mobile phones, in other embodiments the apparatus of the present invention comprises web based portal logins with hyperlinks, and in further embodiments, the apparatus of the present invention comprises linked and encoded SIM and eSIM cards in non 5G mobile cell phones which are also linked to hardware antennae radio devices.

### DETAILED DESCRIPTION OF THE INVENTION

Introduction to Detailed Description: The Blockchain Healthcare Cryptosystem invention is an apparatus working within an apparatus based system. The Britannica Dictionary entry of “Apparatus” begins with two definitions: 1) a tool or piece of equipment used for specific activities; 2) the organization or system used for doing or operating something. Deeper dives into the word from other reference sources mention the words “machine”, “means”, and sometimes “software.”

I present the invention as an apparatus because the make-up fits this description. The invention has at its core a graphene carbon allotrope material based chip encoded with the capability to absorb standard integrated Fourier Transform integrated biofeedback signals from skin temperature, circulatory/muscular rhythm, and unique person “positive ID” capable EEG brain waves. The material approach can work with other carbon allotropes or in combination with others. (Examples of carbon allotropes are graphite, carbon nanotubes, and diamonds.) It is also capable of transmitting signals. As such, it performs as an antenna. It is capable of being encoded on a debit or credit card, a SIM, or other physical media. With the evolution of SIM into eSIM, my coding works on a virtualized card as well. The invention also has firmware and software encoding processes that sync from the graphene material to my blockchain and reverse and allows the hardware to be integrated as a portal into it. I call the blockchain the healthcare cryptosystem network and the material acts as a blockchain oracle within the concepts of IoT or the “Internet of Things.” It pairs by bridging between physical antennae on the material and syncing with coded antennae pairs within the software architecture and distributed network nodes.

Viewing this apparatus as the keystone unit which is replicated across a distributed network in a mechanical, machine-like approach, the cryptosystem is thereby created. I consider both the individual keystone components and the system they exist on as one. As in a stem cell of a living organism, the cell alone has no context without duplication, multi-cell self-generating emergent properties, and differentiation in the organism based on locality and need. These concepts are built into the cryptosystem.

I combine together hardware, software, and blockchain code and processes specific to the Healthcare technology sector. I optimize the distributed server network by defining a multi-level ledger known in the industry as a distributed “Designated Record Set” that incorporates UMLS (Unified Medical Language System) Metathesaurus reconciliations, HL7 (Health Level 7) standards including FHIR and CCDA, X.12 EDI, and USCDI (United States Core Data for Interoperability) data elements within a blockchain consensus validation protocol which I call PoP or “Proof of Patient.”

This combination of standard based entities working with the new processes invented herein is the core unit of the Blockchain Healthcare Cryptosystem. As mentioned, I call these units the “keystone” components and they are duplicated across the system. However, even as this unit is complete in and of itself, it allows for API or other portal-like standards based peripheral connectors of hardware or software for ancillary add on functional entities or programs. These can be produced by third parties using readily available components or with the assistance of Artificial ChapGBT-like Intelligence applied to my “How to Build” approach. In fact, a significant portion of “Component Summary Description Topic 2 on Blockchains” in this detailed description is written describing third party blockchains and how they help formulate and interact with the healthcare cryptosystem blockchain.

In figures accompanying this invention, I use AI generated responses to define the steps necessary to create schematics that are usable by the average CHIP designer and producer to create components. It is my view that ChatGBT-like statements of design can be viewed by many as only regurgitated processes based on already existing known approaches from human records and therefore may not meet the unique and innovative patentability requirements of a new invention. However, it is the bridge between the components and the overall organization of them that only a human can create as I have done and present. There has been progress towards the use of AI in chip design. Researchers at DeepMind are utilizing generative AI neural networking with reinforced learning in AI chip design. It streamlines the human process and reduces the time investment but does not eliminate the human element.

The success of my healthcare focused system is not defined by the efficiency of the intersystem processes but rather by the emergent capabilities triggered by events performed by doctors as the components of the design, and acknowledgement of the ever-changing touch of humanity.

I use the same approach in presenting to the reader of “How to Build” in the creation of an Etherium ERC20 Token. In my provisional application which is referenced in the Application Data Sheet, I had originally included a step by step program code file to create a token for a “GO” compiler. It became quite clear to me that this token creation process is not unique and anyone can find the steps to build one. I therefore suggest that a program such as ChatGBT be utilized to find common ways to create the token and I have removed that figure from this non-provisional patent application but have retained the steps involved in this detailed description.

As an analogy to a “Swiss Army Knife” or a “Black Box” tool kit for adding peripheral components, at the core, I view this working apparatus as the designated holder of the representation of a healthcare patient designated record set. It is an inventory of everything discrete in healthcare built into a ledger (open or closed) that doubles as an actionable tool and updates all related ledgers in real time upon any transaction (Read, Update, Add, Delete) done upon any part of it. Once built and populated, it always exists. In one embodiment, it fits onto existing computer networked servers in the IPFS distributed server environment as well as proprietary networks with the proper API's. There is no need for further discrete collection processes; it builds upon itself. I expand upon embodiment below.

The reader will find throughout these writings that I interchangeably use the pronouns “I” and “we” in my descriptions. The invention and concept is submitted and owned by me as an individual and not a group or company. This is referenced in the Application Data Sheet. However, I also hold the title of president of a company known as Suncoast RHIO in Florida and in the preferred embodiment and proof of concept, as with other embodiments described, Suncoast RHIO will be the organization that will be used to demonstrate the first working use-case.

This description will lay out the details of the Blockchain Healthcare Cryptosystem invention with its emergent-capable software, servers, and methods with example through preferred embodiment of a common “use case”. Healthcare is a complex environment addressed by the invention. Healthcare and the technology that supports it as well as the functionality of this invention are meant to work by drawing from the seamless utilization of interacting components. The description begins with an introductory approach that includes a best mode embodiment. This embodiment is at first presented in summary form and briefly mentions other embodiments that can be used. It is later expanded upon in the Detailed Description in step by step format.

After the summary of the preferred embodiment, this Detailed Description of the Invention is then broken down into two Division Segments with the first (Division Segment 1) being the invention itself and a healthcare industries' historical “Deep Dive” that led to the software architecture. This is complete with its own Table of contents and is then followed Division Segment 2, which is the step by step detail oriented segment of the best mode embodiment mentioned and addresses how to make and use the embodiment as well as other component technologies that would support other embodiments.

This is a technical invention which aligns to new and unique apparatus tools and business systems and methods for healthcare operations. The physical innovations are explained in detail. The invention also leverages human organizational components that work together to form an improved Health Care System segmented by demographics and local or regionalized social determinants of health.

The issues that the invention addresses surround the difficulty in dealing with existing multiple incompatible software systems, inaccurate patient identification, ransomware, and incompatible approaches. These issues are due to a lack of a format for a standard record designation for patients and entities which we address and normalize via the Web3 Blockchain approach. This apparatus along with systems and methods works as a cohesive mechanism and solves these issues while at the same time taking into account the nature of constant change in the industry. The best mode embodiment is therefore applied to an example of a high priority use case and it can be utilized by researchers or government entities and consumers in transactions with their providers. Other embodiments are applicable to expanded patient use and to providers and payers.

The technological components of the invention that are useful, novel, and non-obvious, are made up from A) Our “Internet of Things” or “IoT” antenna's and chips created and coupled with paired software on the blockchain and B) new and integrated software built into our emergent blockchain tokens, API's, and smart contracts (which can simultaneously exist as firmware based “Smart Contracts” for the pairing). Together, these technological components complete the apparatus’.

In some embodiments, the apparatus of the present Healthcare Cryptosystem invention comprises linked and encoded SIM cards in 5G mobile phones, in other embodiments the apparatus of the present invention comprises web based portal logins with hyperlinks, and in further embodiments, the apparatus of the present invention comprises linked and encoded SIM and eSIM cards in non 5G mobile cell phones which are also linked to hardware antennae radio devices, and in further embodiments, the apparatus of the present invention comprises blockchain tokens generated by the present invention's smart contracts which are encoded on graphene chip cards and synced to Web3 based distributed networks and file systems using healthcare HL7 standards, and in further embodiments, the apparatus is comprised of hardware, systems, and methods which in part, can be applicable to the finance industry (Reference to claim 7).

Preferred Mode Embodiment Summary (Detail Steps are found in Division Segment 2).

The use case focuses on healthcare payments with real time provider quality scoring.
1) Build a standard ERC20 Ethernet Token.
2) Build and incorporate Web3 network file on IPFS with blockchain domain name.
3) Program Smart Contract coding on both virtual metaverse-like web and encoded physical antennae chip.
4) Use API bridges between environments in the form of SIM Oracle on 5G mobile.
5) Collect patient consent and upload health record to doctor and hospital cohort.
6) Complete provider encounter and payment with SIM phone with face-sheet data.
7) Provider billing utilizes this blockchain via FHIR through HIE.
8) Tokenized data performs through Suncoast RHIO oversight, pharmacies, payers and in Metaverse mirror database element for consensus.
9) Tokens use smart contract to interface with CMS and Suncoast RHIO Qualified CMS MIPS Registry for quality score results.
10) Validation of data with our new and unique PoP protocol (patient, provider, pharmacy, payer) after traditional PoS is performed.
11) Reward of network tokens backed by commodities or Fedcoin. (FedNow)
12) Patient balance reduced and provider balance increased in distributed ledger.
13) Provider contracts with payers and others updated on this blockchain.
14) All participating providers can now view up-to-date elements of every patient regardless of location.

As mentioned above, a quick summary of alternate embodiments of this use case includes A) Suncoast RHIO portal login with direct SSO bridge connection via hyperlinks to IPFS through distributed Metamask Wallet; B) Direct graphene antenna card without SIM on non 5G mobile with HoneyBadger BFT radio device; C) use of Emergent Seed (ours) smart contract token and Generica Tokens with optimizing Opportunistic Blockchain to RHIOTOKEN (ours) and creating new and adaptive RHIOCOIN upon validation.

As summary reference of USPTO classes and keywords, the following is provided with fields gleaned from USPTO classes that are related the application but not in total: Cryptography (Class 713); Virtual Machine Control (Class 718); Multicomputer Data Transfer (Class 709); Database Management (Class 706); and Operator Interface as related to Smart Contracts and Tokens, and Crypto Coins (related to automatically generation of column and field layouts in electronic documents for external (human) and internal (API and Oracle) operators (Class 715). Keywords within the patent application such as Health Care Management (USPTO Class/subclass 705/002); Insurance (USPTO Class/subclass 705/004); Discounts and Incentives for use in creation of personal concierge plans (USPTO Class/subclass 705/014); and Electronic Shopping so as to match systems and methods to consumers and for network emergent token creation and processing by smart contract (USPTO Class/subclass 705/026).

Divisions and Segments

This next section is an expanded overall invention description covering history, current need, and technical architecture and is made up of Division Segments. 1) The overall description is presented in High Level and Moderate Level; 2) Detailed technological step by step embodiment description mentioned earlier and deep dive description of other components of the invention illustrates use in other embodiments.

Overall Document Layout:
Division Segment 1=Detailed Description Overview;
Division Segment 2=Step-by-Step detail of best mode and other embodiments;

Division Segment are presented in:
Section A=High Level
Section B=Moderate Level with 4 Parts within these Sections

Note, only Division Segment 1 contains a Table of Contents

Division Segment 1

Table of Contents
Detailed Description Overview; Section “A”—
High Level, Parts 1 through 4:
Part 1—Preamble to the Main Section, What is the Invention?
Part 2—Component Discussion
Part 3—The Road Before, Leading to the Current “As Is” State
Part 4—Moving Towards Solutions
Description; Section “B”—Moderate Level, Parts 1 through 4:
Part 1—The Architectural Perspective
Part 2−Conceptual Architecture
Part 3—Patentability Proposal Overview
Part 4—Innovation and Government/Payer Readiness

End of Table of Contents

Part 1—Preamble to the Main Section, What is the Invention?—High Level

Jim Cramer of Wall Street and media fame once responded to an AARP interview question on financial matters. He was asked to address what else, beyond sound investing was an important message that Jim wanted to convey to readers. His answer was, “I got hurt this year. I hurt my back. I wasn't able to walk for four days. I had thought I was indestructible, and I didn't have a plan for not being indestructible. I did have health insurance, but I think that people have to be mindful that nothing works unless they are in good health. So, sacrifice everything first to be in good health, everything. That should be your number one priority, because money you can make or lose, and hopefully make. But if you do not take care of your health, then it's all over. I can't help you”. (AARP Bulletin, December 2021, page 14)

This invention lays out a solution that addresses the problematic U.S. healthcare domain of technology related systems of computers and software that are incompatible, dysfunctional, inaccurate, and unreliable. It addresses the need for people, companies, government, medical providers, and others to pay for healthcare, track it, keep it protected and secure, and optimize it as one unified system replacing disparate islands of contradicting information. The major issues requiring attention are information overload; disparity in data approach across care settings; inconsistent processes; and lack of secure and reliable record keeping.

Too much accessible yet globally incompatible information, replete with non-conforming access mechanisms, is demanding people's attention today. Taken as a whole, interactions over so many “un-synchronized” computers, telephones, smart phones, and other units, involve using unnatural interfaces and they produce over-stimulation and wrong results. This is often followed by anxiety, especially when one's health or the health of loved ones being cared for, hangs in the balance of performing technical steps as system interactions in the correct way. We all live within our own current understanding of the larger pie that overlaps across many more systems that were once barely manageable. Now, with constant change, the normal activities of living that are dependent on these systems of care have become unmanageable; especially when the delivery of care expands beyond the locality of the patient and involve social and demographic aspects and technology adeptness.

At a high level, this invention can be reduced, summarized, and described as a combination of two interacting categories: 1) a Technical Invention−new software and new IoT firmware embedded devices, and 2) a Process and business method to integrate and manage them. From an introductory perspective, we combine new Blockchain and Metaverse related software to match to real world existing healthcare systems and applications already in use. We do this with new and unique Internet of Things (lot) Antenna technology working in concert with mapping software. The antennae exist as both physical antennas on credit or debit cards for example and have programmed paired antennae and synced counterparts in a virtual and mixed reality programmed into a mirrored networked Web3 database arrangement. The Metaverse referred to is also called a mirror database as described below but at a conceptual level, Metaverse in the context of the invention, is the collection of one focused electronic atmosphere around the physical world. So, it may include social media, Blockchain Web3 networks, gaming systems, and electronic news and groups. To see how items that are represented in digital form in this electronic atmosphere around us, we can see how both negative and positive reactions in the physical world of social media are born in this atmosphere, or are inherited from the physical into the electronic human centered atmosphere, manipulated or normalized there, and then sent back to physical life where they have real physical effects for example. (Pokemon-Go, Jetpack, Digital Clothes)

Our mapping approach utilizes both “list based” conjunctive database processing as well as traditional electronic virtual “GPS like” mapping software. Just as our IoT antennae exist in pairs with one in physical existence and the other in 3D print code that is executed into our “Metaverse like” electronic world copy, we integrate our physical and virtual antenna pairs the same. We utilize the built in semi-conductor processing capability and also integrate that code as a part of the smart contracts. This brings the contract software to an object inheritance state with this advanced antenna embedded code. It allows the smart contracts and the antennae and all electronic connections to the antennae, to be located and matched. In the physical world, GPS radio locates and confirms antenna location, frequency, and attributes with our antenna graphene spliced material tagging resources and matching to their virtual world counter parts. Internally, the GPS process is simply replaced by coded programs acting as GPS cloned processors similar to orbiting satellites but now in our electronic atmosphere. Because we use blockchain, we apply the same type of validation in segment blocks on a ledger where instead of validating financial transactions as is done in a Bitcoin or similar currency reconciliation, we are validating the proper contracts and that their tags are correct and we do not perform the commit portion of the match until validation of the matched antennae by our own PoP (Proof of Patient) protocol. Tags are key to our process and the antenna material is the actual portal key to our Metaverse like Mirror-verse-More on this later.

The inventory of parts are: IoT devices and IOMT (Internet of Medical Things) used as Blockchain Oracles with entry trigger points; “Web 3” influenced programs and software in the form of Blockchain smart contracts and “wallets”; Health Level 7 assigned Object Identifiers (HL7 OID's); emergent and unique “seed” blockchain tokens and; the human element of localized organizations.

We introduce our own unique “Blockchain Tokens” that interface independently and execute computer programs. This also means, our primary token when launched, creates other “template driven” and pre-defined tokens as well as new emergent tokens when conditions warrant. The generation of these “new” tokens and contracts is triggered by the events surrounding token transportation through ever changing cells of locality as opposed to “set in stone” expectations of functionality. This is similar to the natural and unique formation of snow crystals (snowflakes), where no two are alike through the dynamic climate of a storm. In our case, new tokens do evolve similar to natural processes. Some may ask, “Why have smart contracts always being newly created in an ongoing emergent process?” The answer lies in the inherent evolutionary mutation rules of nature. Rather than create contracts to attempt covering every unanticipated arrival of a new use case, our approach creates random contracts that sit in wait for appropriate use cases to process; if none arrive, it deletes the obsolete ones with time and use parameter settings. More detail on this process follows in the “Moderate Level Listing of Major Components” Section “B” and in the “Division Segment 2, Building the Best Use Preferred Embodiment.” A summary listing of innovations of the invention can be found in FIG. 1, and relates inclusively to claims 1 through 9.

Inclusive with “Smart Contracts” and “Oracle” technology is software and firmware that uploads health data from traditional systems. Programs perform normalization calculations in a database mirrored in a Web 3 superstructure and download the normalized data back to target systems and on people's devices. The primary Blockchain token is designated the “RHIOCOIN” and it triggers the referenced emergent coins later detailed. RHIOCOIN represents more than a single program. (Note, this proposal is not a proposal for a cryptographic currency such as Bitcoin, even though it can act as a medium of exchange; instead the system augments medical providers’, their facilities’, and patient records and payments using many forms of tokens and currency and other “trade-based-mediums-of-exchange” including up and coming CBDC's (Central Bank Digital Currencies).

Our integrated system represents the genesis of an interdependent blockchain working within the standardized platforms of Ethereum, Cardano, Polkadot, Secret (formerly known as Enigma and aimed at bringing privacy to Smart Contract platforms), Bitcoin, and others. The result is a Healthcare Cryptosystem that in itself acts as the architecture exhibiting various degrees of plasticity. This plasticity is derived from the RHIOCOIN token through repeatable processes over time. It is therefore a dynamically perpetuating process with coins—or Tokens, and contracts re-emerging constantly anew through natural and mechanical change that is represented from the “parent” data. Along with all of the components, the RHIOCOIN represents an integral entity that forms the keystone of our blockchain based healthcare cryptosystem. RHIOCOIN is the “seed” token that results in the formation and the content needed for all of the secondary tokens databases formed in our self-configuring system.

RHIOCOIN is derived from the term RHIO which is an acronym for a “Regional Health Information Organization.” In our context, a RHIO acts as a human support entity organization as well as a blockchain with a dual job to monitor though governance and to perform actions to insure mining and minting occur through automated and human coupled processes. The RHIOCOIN domain is www.RHIOCOIN.Crypto. The website for this domain is under construction on the “IPFS” distributed file system using the “MetaMask” secure distributed wallet though many of the most common wallets are supported. The building of the RHIOCOIN; it's secondary layer of support tokens (Generica; HealtheHub; EmergentSeed.crypto; StemToken.crypto; and RHIOToken); other 3^{rd} party tokens; Non-Fungible Tokens (NFT's and nested NFT's); RHIO's own “Smart Contracts”; the HL7 FHIR and OID (Object Identifiers) with X.12 mappings; the trigger points; the built-in medical quality measure comparative analytics tool; the firmware and hardware entities based on IoT mappings (the “Internet of Things”); the software and hardware Oracles; and more, are all defined in the detail of the embodiment section (Division Segment 2). Examples of real world use cases are included.

It is important to state even at a high level overview, that this invention is not simply an abstract idea formed from existing collections of prior art; this invention is unique and is commercially viable. It uses machines, in the form of computer servers and nodes that process unique data inputs using proprietary “Smart Contract” programming (including programming for paired virtual Antenna Oracles). It creates outputs that are unique and encrypted for use as inputs either into other networked database servers or as end-user interfaces (including portals, smart phone displays, and hand devices) in themselves. The code to operate these accessories in a “mirrored database environment” is the same code that works in physical world programs and IoT devices.

The derived “Healthcare Cryptosystem” is presented in deeper levels of detail as we move through the description. As stated, it is a combination of computer device hardware, software code and programs, and human interfaces that together creates a digital gestalt overlay to our current healthcare system. Its processing functions are to: first, “map the physical world healthcare system into a networked and distributed database”; second, to create a real world reflection of what is in the real world reflected in this now mirrored networked database; and third, to allow changes in this mapped and mirrored database that reflect real actions and real outputs. Upon informed consent and acceptance, the mirrored database overlays the physical distributed networked databases via an historical ledger. This bidirectional aspect of the interacting databased is accomplished via programmed electronic bridges in the form of previously mentioned Blockchain Smart Contracts and Blockchain Oracles in the software code and in the “Internet of Things” firmware. In fact, the invention addresses lot antenna devices which are coupled and paired between physical antennae and mapped to software virtual counterpart antenna programs. Antenna functionality also exists on physical graphene allotrope on chips and cards that can be mapped to DNA as sequencing for another level of validation. This achieves synchronization and preservation of state changes following a process based on programmed instantiation.

This synchronization and preservation into “one” system is always in check. With oversight and auditability, the system allows changed entities in the mirror database to bridge back to physical assets on the IoT enabled deices to reflect changes and enforce consistency, interoperability and unity. One of many trigger points can include the end user themselves. In this use case, when the end user is satisfied that changes are truly reflective of the reality of the health care episode(s) will the update occur always with an historical reference allowing correction if errors are later found.

Not Quite the Metaverse, a Metverse-Like Mirrorverse

The healthcare system that currently spans our country uses multiple technologies and applications that do not interoperate. This adds a significant layer of confusion and introduces a lack of synchronicity. There exists no one database where all system interactions are logged and their effects on other parts are seen as one. However, there is another grouping of systems that can normalize the disparity because they are created to reflect others. Though clichés are not the focus of this proposal, the coherent system referred to today is known in gaming circles as the Meta-verse. This term, used in our case, only serves to give the reader a sense of comparison and is a reference point.

By using blockchain and various pathways into the databases that run in a Meta-verse like environment, we can utilize the consistency and unity of a common software approach or architecture—Make changes on it, and send corrected environment back to the physical ecosystem. This is because the systems that support Meta-verse programs and games are new and consistent with all other games and programs in order to support users going from one game or program to another without having to reproduce or reprogram themselves or their VR, AR, or other ancillary devices.

Program languages such as solidity and python are common across Meta-verse and blockchain and programs are made with interoperability in mind. This proposal is not suggesting a “game playing” field in virtual or augmented reality, but by using Web3 interactions, it helps explain the approach better than if a Meta-verse concept did not exist at all.

API's created in this proposal will normalize the physical world data when copied into what this invention calls a “mirrored world”, and keeps the databases in check. RHIO “Smart Contracts” are the avatars and these avatars, which are really programs and in some cases physical antenna cards acting as Oracle IoT smart contracts, are bidirectional in their interactivity. By having the “Smart Contracts” living in both the physical world and in the “mixed reality” (MR) world the invention executes in synthesized, synchronized, and parallel fashion.

They uptake bad data, fix it in blockchain environment, and send it back corrected. To avoid confusion going forward, we will refrain from using the Meta-verse terminology and instead use “Mirror Database” or “Metaverse-like mirrorverse” where possible. This is the point of entry for the invention.

The missing ingredient that today affects our physical world health care system cannot be found in chaotic, dysfunctional, and absent record of changes with “too many” systems and actors within them. We address this with our “Overlay” mirrored database, similar to the “meta-verse” approach but instead of with avatars and programmed events made up for a game, we have events created in the real world and represented metaphorically. As in the mirror database of “Avatars”, there are now representations of real people, patients, doctors, standards, systems, facilities, and “smart blockchain contracts.” These have a foot in each world and in databases where we can see, not with visors or gadgets, but purely from computer to computer code, and both as one record of data.

This provides us the benefits of combining unified interactions and their effects and allows adjustments in the flows and connector points as seen from a bird's eye view. In effect, the Meta-verse mirror database bridges to real world entities. Banks have used this approach for years but under different names. As an ex-technology banker and CIO, this method was by this inventor as an approach to keep an untouched customer legal record separate from any portal copy of records or transactions used by bank customers. We also include the healthcare industry working quality measure derivation and reporting for medical providers much like credit scores complete with audit responses and networked based health data exchange. Experience in clinical functions built into the invention include patient Direct Care for social determinants of health and certified electroencephalography psychophysiologist (EEG and qEEG) work.

Part 2—Component Discussion—High Level

The invention has multiple points of entry. These points, existing in many forms, act as triggers. The method utilizes “data altering” and “data access” processes via mappings to the vast array of standardized systems and medical nomenclature. The connections to this “Overlay” mirror database include: institutions, companies, and people. The system is interoperable on many levels and utilizes traditional systems combined with blockchain technology. It gains the advantage of decentralization, secure transactions, safe distribution, and non-mutable ledger record keeping, and importantly, with social community involvement (represented as social governance and management via distributed structures). This is a systematic method that integrates with blockchain frameworks, including its own, in an architectural approach.

Listing of the Major Components

At the highest level, the overall project can be viewed as a collective. The major components and the primary functions are here presented.

The Rhiotoken Blockchain Token and Secondary Support Tokens
Secondary tokens are Generica, HealtheHub, EmergentSeed, Stem, and others (each supported with .Crypto, .Wallet, and .ZIL domains)
The RHIOTOKEN generates the RHIOCOIN as currency

Hardware—
Electronic enabled devices in the form of IoT (Internet of Things) physical objects
An “lot” Multi-function Wallet Card (one of many doorways into the system)—semi-conductor or software (thereby avoiding current and future shortages), and antenna technology.
Other secondary devices utilizing proprietary software & firmware into our lot Network
Credit and Debit cards with secondary “Wallet” capability for access to medical quality of care scoring operations (Based on CMS government MIPS and VBP algorithms for providers that result in near real-time feedback. We also support common Credit and Debit cards.

Middleware (Hardware and Software)—
Blockchain Wallets based on MetaMask and embedded proprietary coded Smart Contracts
A Distributed Computer File System (IPFS)
Mappings to established healthcare standards & software I.E: HL7, FHIR and OID, EHR, X.12
Backup, recovery, security and distributed self-promulgating hacker and ransomware resistant emergent tokens and networks
API access to the databases
Virtual (via code) and paired antenna Oracles

Software—
The distributed networked mirrored database: “The Overlay Database”
Distributed Networking “Transaction Inspired 2 phase commit” software and EDI X.12
NESSI, An analytical comparative statistical software tool based on “Data Envelopment Analysis”
Generic blockchain token inter-activity processes together with NFT tokens and nested NFT's

Human-ware—

Regarding this aspect of the “Human” component, it is wise to always remember that healthcare is about human wellbeing. It is not only about the best care for a sick patient, but it is also about the “Art of the Application of the Fields of Medicine” practiced by the physician and members of the care team.

As per Brian Bothwell of the US General Accounting Office (GAO) in spotlight brief GAO-22-105541, Extended Realty Technologies, January 2022, “Extended Reality (XR) is the overarching term for a spectrum of technologies that link or integrate the digital world and the real world. These include augmented reality (AR), mixed reality (MR), and virtual reality (VR) technologies, all of which provide different degrees of sensory immersion and interaction between the real world and digital content.”

In our case, we are omitting the human sensor aspect, and replacing the user with the concept of a programmable smart contact. Any representation of the real world in the digital world can be accomplished through computer coding. As with the concepts of 3D printing, using computer code to recreate paired representations; this can include virtual sensors, antennae with one being encompassed by our smart contract “avitars”, and even the micro-chip technology and materials themselves, acting as antenna (see Athinia.com and their partnerships with the medical company Merck KGaA and Palantir working with their “Foundry” Operating System) that explore, process, and hold data. These can be linked to medical and biological sequences. Virtual sensors can be used to gather information and accept commands. XR-enabled code helps with reconciliation and to fix compliance and misaligned diagnosis and historical treatments planned or already preformed with their complete history. Multi-layered security in cryptographic technology, cryptocurrency, and AI, supports clinical harmonization within an IoT connected and distributed networks of systems and analytics.

XR could also make more data vulnerable to cyberattacks and privacy threats, which is why the invention introduces a reconcilable paired (or further nested) bridge to the real world. Reconciliation is, in effect, a multiple “Miner Proof of Work” to blocks of data where the miners are people, software, networks, and staked tokens. Data analysis in XR environments might allow new kinds of knowledge generation or decision-making as well.

Part 3—The Road Before, Leading to the Current “As Is” State—High Level

In this section, we discuss the opportunity and a brief history of the evolutionary snapshot of how we arrived at the “now” state of the current healthcare environment we are now in—(Note, this is not directly related to the technical aspects of the invention but forms the basis as to the “WHY” this invention has come about in addressing a broken “AS IS” state):

To say the U.S. Healthcare System is complicated is an understatement. As it exists today, our overall healthcare eco-system exists in a disjointed collection of non-interoperable and almost “uncountable” parts and systems. One finds exceptions within local and relatively small cohorts in physician practices, hospitals, or clinic type entities, but bridges of interoperability to other localized systems under different management or ownership, exist and work only with significant human intervention. The effects of this non-interoperability results in poor care on a macro level, confusion of parties affected, high utilization and unnecessary cost, and disparities in delivery. Information overload has reached critical mass.

Waves of technology innovation have swept over our communities faster than our communities are able to absorb their effects and reconcile with those changes in a unified way. Healthcare is too critical to ignore. Sometimes the answer to an overwhelming existence in a world driven by disconnected technology is to let the technology first play itself out on its own layer before allowing it into our lives.

Technology fixes based on older systems is impossible. The few healthcare technology firms that can afford to adjust their technology to stay on top of the ever changing challenges of meeting compliance regulations and the ever changing computer language and coding differences are limited and constrained on funding. Many firms produce proprietary software systems to retain market dominance and discourage small companies that are software nimble, yet these proprietary systems cannot keep up with changes demanded through “open source”. Skilled employees are far and few between and there are less and less of them over time. When they are hired by established healthcare software firms, their precious time is taken by being engaged in training by their employers in old technology systems sentences these hard-to-find-employees to learn to only work with one program, usually owned by only one software vendor. Skills are non-transferrable.

On a larger global stage, we are living in a new paradigm world of intelligence workers no longer limited by space. The factories of today are not based on foundries of melting iron but on technology savvy workers who are goal oriented each in their own way. By addressing our healthcare eco-system as proposed, it is possible for us to reinvent our factories into intelligence workers making intelligent based blockchain widgets while exporting and importing superior healthcare to countries in need. The U.S. can once again become a net exporter of goods and services while optimizing an American workforce in tandem with offshore blockchain trade partners.

Government and payer funding is also behind as much as customer prices and fees. The costs of modifying proprietary systems are very large, sometimes surpassing over 30% of the annual vendor's revenue, and which are many times simply upgrade and maintenance fees. These fees are then ultimately passed on to consumers. And the demographic of the “up and coming” working age youth who are also conversant with older technology becomes non-existent. It seems to be in vogue to disparage today's youth for not rising to the challenge but they didn't do this; they inherited a broken “Hodge Podge” of systems created by an older generation with older skills from a generation of programming in hierarchical approaches. No one can manage these systems who is not themselves somewhere near retirement age or trained exclusively only on non-interoperable software. The youth of today should be applauded for giving society the answers to work with these systems by replacing them. Even the very young have access to training programs that prepare them for this industry. Blockchain is their gift to us and is why this invention embraces it.

The Patient Effect

Older patients that may be on fixed incomes many times have their contributions and benefits capped. They are unable to keep up with the price of care demanded by health care providers facing their own challenges in paying for software. This drives everyone into a downward spiral of chaotic non-interoperable expensive technology that no one can pay for.

Healthcare in the United States is too expensive. Healthcare Information Technology (IT) is approaching 20% of Gross Domestic Product (GDP) while GDP is made up by 70% of consumer spending. And as stated, healthcare is noted for disparities in care access and for disproportionate distributions of rendered care with expensive treatments. Looking at the breakdown of national GDP another way, that 70% of consumer spend is attributable to patients, yet the 20% of nationwide healthcare as a function of GDP is not evenly distributed to that 70%. In the consumer component, we see that there are disproportions in the distribution of wealth that appear in national healthcare benefits as well.

A short exercise in mathematical extrapolation exhibits this point of disparity between wealth distribution and quality care distribution. Set one factor to represent expenditures and the other to represent benefits. It becomes clear that dollars collected come from “the many” over time while benefits go to “the few”, or the highest bidder in the present. (As with debt where we take from the future to pay for the present; in this case, we take present value of past contributions, such as Medicare, to pay for the higher prices today) This segment disparity creates a loss for the majority of beneficiaries even though that was the segment that footed the bill through program deductions over their lives. For the few, their own expenditures come from other sources of income or dynastic wealth that pay above insurance plan distributions yet are only targeted to them at that one time when desired. They benefit from the paid for research and innovation of the beneficiaries.

Another way to view this relationship is to look at the 1% to 5% of the wealthiest segment in the country needing healthcare and wanting the best when needed. Using personal wealth to utilize, pay, and “augment” the bill, with some small exceptions, to satisfy provider charges. The system as a whole, including the research and discoveries, was mostly paid for by “the many” via tax and entitlement deductions. From a short term perspective, sellers of healthcare, such as physicians and hospitals will allocate services to the few because of the chance to charge higher fees for specialized care rather than have that specialized care available to all and paid for at much lower prices and insurance determined rates. This avoids the care at the capped rates that was planned and determined by insurance and government via contract and avoids having to deal with prior authorization processes; but this is not a luxury that the population as a whole can afford.

The healthcare system and its benefits are finite; there is just so much specialized care available. This leads to scarce specialized personalized care now going to the highest bidder. If anything is left over, it is in much smaller portions of the overall set of specialized care that has already been removed the majority's needs. With less available care left over there is a relatively disproportionate amount to spread to the 95% of the population that need it as much.

The United States has some of the best healthcare practitioners in the world, quality medical schools, some of the most dedicated and caring professionals in the art of medicine, and has superior research institutions and hospitals. These individuals and facilities are as much victim of the systematic failure of an unchecked growing and an overly complicated system as the rest of the population. The issue is with the cohesiveness of the system or, the lack of cohesiveness. We exist within many locality based systems each within frontiers serving various cohorts of patients, and each locality performs efficiently in itself. But in the scheme of a lack of an overall interacting system to tie them all together, the failures exhibit themselves at every turn. That is an issue this invention addresses—“Interoperability”- and not just limited to the technological ecosystem

Part 4—Moving Towards Solutions—High Level

This invention tackles the problem and the opportunity, to normalize and simplify healthcare through blockchain technology. The approach embraces interactions of complex and confusing factors handled today by human interventions and computer software incompatibilities and instead converts these “observable disconnects” efforts to a mapped out computer based decentralized and networked databases with clearly defined lines of connectivity. This puts the complexity of the interactions behind the curtain of technological processes. As another emergent innovation made possible by the invention, it creates a framework that provide the ability for everyday consumers to create their own health plans through “comprehensive healthcare personal concierge systems,” comprised of all desired components; doctors, payers, hospitals, Health Savings Accounts, and clinics. Health insurance “payor” companies such as Cigna, United, CVS, Blue Cross and Blue Shield, and others, are moving into these technology arenas via “Virtual-First” Telehealth plans—though not quite there yet—complete Blockchain “To Be” environment. It is a significant step away from “physical only” patient visits. Even as far away as Dubai, “Metahero Inc.” is creating Meta-verse physical portals. People can choose these arrangements. This freedom of choice trumps institutional choice made for us by others.

Where many people and families living in the upper echelons of wealth utilize concierge arrangements, the majority of Americans find these arrangements unaffordable. In addition to demographic factors, a large part of the healthcare affordability issue is directly related to unwieldy healthcare processes and the lack of interoperability of software.

This invention and the concurrent Blockchain project along with it, is the first step towards the democratization of the healthcare system. It works by individual choice instead of insurance company rate sheets and limited choices of credentialed doctors in a health-plan's approved list. It allows the mix and match make up of a personal or family concierge system to emerge from all available components existing in the eco-system. It does this by identifying the fungible components in the current system and normalizes them as interchangeable components. In this arrangement, the only non-fungible items are the patients themselves and their personal journeys and experiences leading to their needs at the moment of care. It is a journey of “Digital Transformation”. This then becomes the ever-changing patient and consumer individualized Health Plan formed as needs dictate via ACO (Accountable Care Organization) governance and member influence; an “Everyman, Every woman, Every Family Concierge!”

Presently, there are scant inter-operable processes. Clinical processes that interact smoothly with administrative and billing processes using computer software programs and packages don't communicate well with each-other. Siloes of different products doing different things and working in different ways are all promising to deliver on the stated goal of interoperability but are failing. This failure is not from lack of trying. For example, just in the way of government incentives, billions of dollars have been spent to address the issue, but there is a long way to go. Other than software, there are many other entities interacting within the current eco-system such as: insurance payers; provider types; practice environments and locations; competing regulations on the State, Federal, and local levels; various coding and payment systems; and many ancillary entities and functions. This invention takes a step towards solving this disjointed problem and is why we start by applying blockchain through mapping relationships.

This invention creates a total “Healthcare Cryptosystem.” The “Healthcare Cryptosystem” introduces entities working within the realm of existing legacy computer systems and human workflow processes. From a patentable perspective, it is seeking Utility Patent approval for the Healthcare Cryptosystem.

As a whole, a Cryptosystem, as the name implies, operates as a system with interacting components of processing units, inputs, outputs, and end users. All components that fall into the patentable realm are introduced in this invention as being new, unique, and non-obvious and within the collective, they are interspersed with some existing prior art. However the Healthcare Cryptosystem collective when working as a unified system, is commercially viable itself, solves a number of problems, and can be used by those in the industry with average skills. It has both software and computer processes that utilize machines (computers) that perform Transformation, I.E., Turning one set of things into another. Taken as a whole, each is explained below and in greater detail later in the document.

Part 1—The Architectural Perspective Segment—Moderate Level

Our physical layer describing the architectural foundation has multiple components In addition to hardware components made up of IoT (Internet of Things) devices and modified payment credit card tools which are both described later, there are 2 additional architectural sections in the Middleware and Software components: 1. “The Medical Hub Exchange Gateway” comprised of these components: A) The “Overlay”; B) The “Viewer”; C) The “Mappings” that are built within unique “Smart Contracts”; D) Unique Blockchain “Tokens”; E) Healthcare Cryptosystem Blockchain “Oracles” (another bridging between physical and mirror software); F) “Foundational Value Extraction Mechanisms” for Collateral Coins; and G) The “Passport”.

The next and second major section is: 2. “The Bundled Hub” that collects and then creates dynamic collections of 3 rd party and already existing Block-chain external tokens and creates interoperability by taking outputs of each token's respective functions via another “Smart Contract” we call a “mesh token”. This allows all of the collective tokens in the mesh to act as an ever adjusting single token unit within the Block-chain of the Healthcare Cryptosystem.

New cryptographic tokens or coins are introduced on a regular basis with applicability and uses that will have attributes that are “inheritable” for use in the Healthcare Cryptosystem for any need, predicable or unpredictable, that may come up at a specific time. As of this writing, there are new cryptographic tokens that aim to use blockchain to create decentralized IoT (Internet of Things) platforms such as IOTX from laTex, would help our healthcare cryptosystem software map from the cloud to physical items, perhaps by barcode or chip. There are also new tokens that fall into the so called “Jetpack” category, again facilitating a mirror image of reality with parallel software. Though Jetpacks are based on games, the coding is applicable to non-game situations as well.

Another example is the 3 rd party token “DOT” where parallel blockchains known as “parachains” can link disparate blockchains together. This brings real world use cases into the total cryptographic blockchain world as does the token known as ADA. There already exist many tokens that address many needs as they occur and new ones are always being developed by the free and innovative developers worldwide. There will always be a need for more developers as needs change. It is impossible to predict each upcoming future application and tool that the Healthcare Cryptosystem will need in every case. This statement supports the need to create the invention in an environment that welcomes and rewards innovation. It must be architecture based as well as have the ability to support exiting legacy standard software and networks. The idea is to embrace change and inherit appropriate development as needed. This in itself parallels the changing healthcare eco-system we all use and depend on.

The Medical Hub Exchange Gateway, referenced on the previous page, contains commercially viable entities within it. It is made up of unique smart contracts (Both existing pre-programmed and new emergent generations), specialized tokens, credit and debit card bridges linking financial and administrative functions to real-time medical quality scores, a statistical comparative analytics module (Internally named “NESSI” for Nested Envelopment Statistical System Interface), and mappings to universal healthcare standards. This Hub is the working component within our healthcare cryptosystem, much as an engine is the primary component of various machines.

In this component, the “Exchange” portion refers to the many programmatic interactions that flow in and out of the distributed databases, dynamically modifying the states of the distributed networked servers in “near real time”. The “Gateway” portion refers to the many types and points of entry including related processing software acting as “two-way data flow doorways” of data, machine, and human interaction.

This gateway process is designated as the Passport. All of these components work under the rules and mechanisms of our “Overlay” methodology. The “The Bundled Hub” noted on the previous page, refers to programs that encompass cryptographic coins from 3 rd parties that have “appropriated uses” to the workings of the Healthcare Cryptosystem at the “right time and place”. The “Bundled Hub” is based on database inheritance and optimized to utilize any and all “appropriate use” crypto coins and tokens including those emerged from seed based smart contracts.

A pictorial representation of these concepts can be found in FIG. 2 and relates to claim 8.

Part 2−Conceptual Architecture—Moderate Level

The Medical Hub Exchange Gateway and the Bundled Hub components are expanded upon here for clarity. The collective components act together as the architecture:
1. The Methodology—Overlay (proprietary)
   i. DEA—Data Envelopment Analysis
   ii. The “Viewer” (proprietary)
   iii. The “Capture, Pull, and Display” software mechanism (For Clinical Treatment, Utilization, Historical, Standard of Quality, and Cost fields)
2. The Mappings1—Health care operational entities
3. The Mappings2—Technology Standards (HL7 CCDA, EHR, OID, FHIR, ANSI X.12, CAQH, USCDI)
4. The Mappings3—The External “Pull and Inheritance” software interacting mechanisms
5. The Blockchain Proprietary Tokens (RHIOCOIN, GENERICA, Health1Hub, GenericSeed)
6. The Blockchain Smart Contracts (proprietary—emergent and other)
7. The Healthcare Cryptosystem's “Blockchain Oracles” (Also related to IoT hardware, “Proof of Network”, radio nodes similar to “Helium,” a third party token mechanism)
8. The “Triggers”—Internal Mechanisms and Catalysts
9. The “Commodity based collateral Coins and Tokens”—Reserve of Value without Dilution
   a. Collateral Coins
   b. The “Mechanism to Extract Foundational Value and Collateral”—instant execution
10. Passport—Facilitates movement; data updates via the token network
   a. See “Emergent and Seed Photon/Biological Mechanical Model” analogy.

The components of “The Bundled Hub”:
11. The “Mesh” token creation program for 3 rd party Blockchain tokens

Our evolving healthcare cryptosystem addresses interoperable processes and define how the unique computer programs in the network interact within the present day existing physical healthcare environment and existing cryptographic block-chains at specific times based on need in a dynamic ever changing environment. Both Oracles and IoT components are critical to this bridge between a cryptosystem and physical based entities.

These Conceptual Architecture components above are elaborated upon below in the section designated as “Expanded Descriptions of Conceptual Architecture Components”

On a General level, the invention uses this conceptual architecture described above, to execute the systems processes triggered by real physical machines/computers outputting data and generating additional data and programs. It performs this over networked distributed databases. Outputs are produced from a combination of distributed computer software programs and by human interactions. These utilize industry wide consistent and accepted methods and software bridges to mapped entities. In fact, a key component is the mapping capability in concert with transformative activities across many health care standards and applied to physical units, treatments, and nomenclatures.
The bridge to the system is accomplished by hardware devices enable with IoT technology
Oracles exist in paired instances in real and Metaverse.
Some of these Oracles are antennae, either in virtual code or real and may exist in materials
Similar in design approach, compare to meta-verse like “digital clothes”
Antennae also act as “Wallets” and the process of calibration performs “Proof-Of-Work derivative consensus known as Proof-of-Stake and Proof-of-Authority.
The tokens created are cryptographic that are assigned “Smart Contract” attributes
It is hosted on cryptographic distributed databases including the IPFS distributed File System
The preferred cryptographic secure “wallet” is the “Metamask Wallet” (all wallets supported)
Tokens utilize .crypto extensions and work within the “Unstoppable Domains” website engines
eGovernance Platform and security layers addressed by “Hyper-ledger Fabric” and “FHIRChain”
“Tokens becoming Other Tokens” processing is governed by “Passport” and “Mesh” based on source and target content as described earlier in Conceptual Architecture.

In every cycle of either on-demand or event-triggered processing or of cycles within larger cycles, our cryptosystem creates “one of a kind” dynamic, encrypted, and unique blockchain tokens which may go on to be inputs to other process; be an end result themselves; interact with or actually become cryptographic smart contracts; or are blockchain Oracles themselves.

The various mirrored database derived outputs can be generally thought of as Units of Health. They are appropriate to the “needs, consents, and permissions” of the user (person or machine), and available at the “just-in-time” moment to those entities with the legal consent and the need to see, know, or ingest them either individually or in groupings. It is a clear and focused picture in ever changing processes through data representation. It creates an overarching interacting structure to the entire healthcare collection of systems that was never there before.

To recap, real world non-interoperable and out-of-sync system components are gathered by various Oracles and copied to a Mirror Database via a Trigger event. These include NFT's which are encrypted. Once in the mirror database, standardized “Metaverse” programs including antennae and internal wallets (normally in use to synchronize different games or experiences in augmented reality), normalize all components with correct and interoperable database updates. At the moment these updated components are normalized, they are copied back to the real environment with interoperable and confirmed data in the physical world. Specifically, this invention consolidates decentralized networks of a multi-coin cryptographic for a healthcare based eco-system reflecting established interacting entities. Software bridges; gateways; trigger points of network entry and catalysts leading to processing events; mappings; and blockchain related software fits within an integrated technology “overlay” methodology. By intersecting clinical health care expertise, computer technology and engineering, and Blockchain, this invention ushers in a new industry in our time.

Expanded Conceptual Architecture Components

In describing the architecture, it is prudent to keep in mind the contrast we introduce that is derived by components of the traditional healthcare system. In the current “As Is” model, there are different electronic medical record products, use cases, and systems. Each has their own language or approach to populate standardize fields in vendor defined meta-data format. Our approach applies “blockchain algorithm based processes” to solve multiple technological problems that exist in conventional industry practice.

The Methodology—“Overlay”

The Overlay Methodology defines the dynamic database rules and standards approach in utilizing the ever growing, permeating, and health “unit” adjusting mechanism. By “unit” in this context, this is referring to the concept of localization and boundary size dynamics of the domains and regions of view by the user and system processes. In this digital version of the proposed system, it is pegged to physical health assets (e.g., Medications; DME; EHR; Codified Frontiers; Patients; Physicians and Providers; Groups; Payers; Hospitals; Hospital Networks; Medical Homes; Government Agencies; Regulatory Bodies; etc.). The unique mappings are discussed in the “Mappings Section”.

As a systems development and ongoing methodological approach, it follows tried, true, and proven historical design parameters and standard system life cycle traditions. It is inclusive of phases and embraces recycled jump off points to new innovative applications. This happens when the development cycle is marked by the end by goal attainment and acts as the beginning of new “next” cycles of use cases. As a distributed de-centralized environment, it may seem to an observer that there are of unrelated efforts simultaneously occurring, but upon closer look, one can always identify within the streams the process units of Analysis, Design, Coding, Testing, Implementation, and Operations. It is within this structure that unique inner workings take place.

The Overlay Methodology defines how development of smart contracts, network interfaces to Oracles and overarching architectural rules on languages, security, testing, and parallel processing is done. It also defines health related working components in a standardized topology, not by place or process or time, but by the topology of events over time and place, overlaying each other as progress occurs

Examples of related data collections and data driven processes that could be addressed include:

Statistical Cohorts (See NESSI)
Quality Evaluation and Comparison based on proven benchmarks and specifications
Inventory Ledger for Networked Record Keeping and Identification
Biotechnology hardware and scripted chemical based innovations
Risk management Interactions based on “Joined” Database interacting entities
Research Groupings/Grid Examination of hidden SDOH (Social Determinants of Health)
Ransomware Threat Capture and Reduction Programs
Health Savings and HRA Accounts
Health Insurance
Barter or Other Currency

There are numerous parts of the healthcare industry that are still using technology systems that are outdated. Only 53 out of 195 countries have a “good” health care index score according to one well known global ranking system. Having a unique methodology to manage the network anchored in traditional approaches helps us to stabilize and test the concept, observe and increase our own index score, and adjust by culture in a naturally global roll out.

Passport Oracle

A passport approach is used by the system itself allow entry to cross-mapped standardized systems that currently exist in non-interoperable states. It is based on a second layer of encrypted private key working with a public key value known internally on the nodes. It works with Web3 distributed data base tools and re-purposed X.25 packet switching technology.

The passport concept is not only for the people and patients that can use multiple services but for the Smart Contracts as well. It interacts electronically via enabled lot radio devices, antennae, and wireless signals following interconnect standards being developed under the SOSA and VITA organizations, and/or via barcode. Mapping is a key to the functionality.

See FIG. 3 for listings of “Cross Walked Mapping Entity Systems” (Five Lists Notated A—E). This figure relates to claim 8.

Mapping

Much like an EDI Distributed and data normalizing clearinghouse that have ledgers and combining process algorithms, our networks run deterministically in the context of machines in a blockchain. It verifies and executes contract terms upon the occurrence of predetermined events. Many times, these contracts can be thought of as programmable validators.

We interface with CBDC (Central Bank Digital Currency) as an ancillary mode of payment. Audit, inventory, historical trend, population health and tracking for quality via RHIOCOIN, similar to an SDR approach (special drawing rights) for market baskets, are in line with CMS & Commercial payers through the TIN/NPI unique identifier.

Smart Contract Emergence

Web3 Smart Contracts with emergent coded capabilities and seed Tokens are programmed utilizing neurological techniques as inspired by evolution and natural selection projects. The invention uses like NFT contract genomes and patient representation to control this process. It allows contracts to self-replicate based on need in the system of required patient identification, patient processes, patient history, and specific and unique longitudinal patient activities required for specific “Patient Care Time and Life Lines,” much like the “World Line” concept of Albert Einstein. This World Line concept is summarized as “To every moment in time corresponds one point of that world-line giving the position of the 4 dimensional object in space at that particular moment. It is a fact of life that not all events in our universe happen concurrently —instead, there is a certain order.”

The invention therefore contains programmed genomes for frequency and applicability acting as switches in our programmed layer utilizing natural selection and neural network programming. This removes the requirement to predict the future need of new contracts and they are generated by program parameters and self-deletion based on usage frequency and randomized code to avoid hacking entry via Oracles.

The programmable approach to emergent contract creation takes into account that each emergent contract has the following abilities: 1) Capability to self-replicate; 2) Having a blueprint template for new contracts that emerge; 3) Having the ability to inherit meta data fields and processes from previous contracts; 4) undergo occasional random mutation; 5) follow a selection method of reproduction similar to biological genome processes in nature. Thanks to work of David Miller on evolution simulators, our non-programmed “creation of contracts/Tokens” follows the observation of “Whatever Reproduces, Reproduces; Whatever Doesn't, Doesn't.

Trigger Points

Trigger points relate to events that may be either inside the software of the cryptosystem or outside catalysts. Sometime these bridges may be events, software, or even hardware that is IoT enabled. We refer to these as Oracles.

An Example of the Trigger Point shows the ability of the software to capture and utilize patient payments when done by credit or debit card, into the “Medical Hub Exchange Gateway”. The listing of services related to the charges on the patient bill includes diagnostic codes and other important clinical and financial information that the medical provider needs in order to send a claim (See Note) to a payer or to a claims Clearinghouse. Payment capture can be done in a variety of ways in addition to credit card. This “Use Case” process is included in our flowcharts and drawings.

We take this trigger point and use the data in the payment, compared against a submission based distributed data base residing on qualified registries as explained below. The insurance claim also goes through normal routes to government CMS Medicare (Federal Payer) and/or Medicaid (Federal and State)/and/or CHIPS (Federal and State) and also to Commercial Payers (using NCQA HEDIS Measures) in addition to a Quality Reporting Registry. The Registry, in this example case, is an organization managed by the invention participant. The organization is a long (since 2013) established registry known as “Suncoast RHIO Registry” (Regional Health Information Organization).

Once data is captured at the point of payment, this triggers the initial and subsequent access to the networked databases and programs described. A cryptographic blockchain underpinning (Not Cryptocurrency) based on various “tokens” and “Smart Contracts” in combination with standardized traditional distributed database technology, carries out the functions intended. With this optional use of blockchain technology, there are supplemental ways a provider may be paid with patient funds that are backed by hard commodity assets or collateral related to the tokens or coins.

The Registry takes the clinical codes from the patient charge along with other clinical data from the provider's systems, and equates them to Quality Measures. It scores for the provider in “Near Real-Time”. Since these quality scores determine the adjustment to the rate of the CMS Market Basket Fee equating payment to the medical provider for Service (FFS), the provider no longer has to wait until the end of the year to see how they are doing and what is their revenue that is affected.

This opens the door to evidence outcome based improvement on clinical care and optimized revenue to the provider. It enables comparative statistical analysis across the country. This statistical analysis and comparative model of the invention uses the Data Envelopment Analysis (DEA) and Frontier Statistical methods. It can be applied across one provider, many providers, or any cohort(s) of any size. It dynamical displays all parameters of comparison. Since there is the option to utilize blockchain tokens, interactions with many other functions in and out of the health care system can be supported. This also includes designated activity in Bio-Technology such as support for research.

By embracing payment models that support current and digital methods of claims payment and traceability, we include upcoming systems of payments not yet in existence such as the CBDC or Central Bank Digital Currencies.

Explanatory Note: A medical claim is what many varied providers at different levels in many organizations do though their Electronic Medical Record (EHR) or Practice Management (PM) software billing function. If the provider is credentialed by the payers, who are the Health Care Insurance companies or other TPA's (Third party Administrator), or potentially even their own Health Insurance Plan if they are a large enough company to do their own claim submission. They then submit the claim electronically via an EDI (Electronic Data Exchange Standardized Software program used by most commercial entities) transaction ID 837. In a second scenario, there are some organizations known as ClearingHouses. Instead of sending the claim directly to the payer, the provider sends the claim to the intermediary clearinghouse service that charges a fee to insure all contextual data is in the right place for the payer. This process is known as “Claims Scrubbing”. If extra documentation is needed to process the claim or if a Prior Authorization is required, an EDI code for attachments (270, and 278) can be used. End of Note

NESSI−Statistical

A statistical comparison software program encompassing the use of DEA, or Data Envelopment Analysis and Frontier analysis.

Part 3—Patentability Overview—Moderate Level

As previously stated, this is an application for one Utility Patent though it covers many features, functions, and processes related to healthcare that can be achieved by adding or removing components. Components are not part of the invention as they are readily available or easily created with standard coding and other existing apparatus′. It marries technology to the real world environment and the Web3 environment with applications built and existing in Web1 and Web2 software applications though a proprietary methodology. It mirrors the real environment with a software architecture that bridges between the “mirror database” programs to real world components. In this sense, as said earlier, we borrow the term “Mirrorverse” as derivative of the word “Metaverse” from gamification to help in our description.

We propose that the invention, as a group of new interacting and unique software programs is itself a unit and an apparatus, with new physical technology acting as oracles, is patentable. It works as one comprising dynamic network architecture with all the parts in tandem while automatically updating “second by second” to reflect changes in the real environment. The “Healthcare Cryptosystem” has ChapGBT AI developed stored and self-modifiable program components patent related attributes:
The invention is used in a way that has commercial use.
It is new, unique, and not obvious to those with average skills in the industry.
It has both software and computer implemented processes.
It is used with machines that turns one set of things into another.
(Transformation)
There is more than insignificant extra-solution activity involved beyond transformation.

We Show:
Descriptions of how the code and processes carries out these tasks.
Portrayal of the design and architecture as a map to outputs of the distributed network.
How the invention embraces new systems that may be introduced in the future.

We Answer:
What is unique about the software
What the software's desired function(s) are
How the software handles information
What the user interfaces are
The problems the software solves

Therefore this is:
More than an abstract idea; not overly broad; is durable; and does not hinder innovation
Has an industrial and commercial way to use the invention
Is used with a machine and it is not just a business method−machine generated encrypted code is unique and derives itself from each smart contract and computer server node and newly invented physical antennae and paired coded antennae built with focus on known methods and coding currently used in 3D printing in order to generate MR (Mixed Reality) virtual code.
Uses encrypted code as input to other computers and processes—it passes the machine or transformation test.
Software and processes are tied to a machine to turn one thing into another but is not restricted to machine use—there is more than insignificant extra-solution activity involved.
Is new, unique, and non-obvious to people with average skills in the industry.
Patentability results from new hardware, antennae oracles, software, PoP methods of validation, and features and functions created using new technology and the unique combination of component interoperable functionality upon existing technology that emerges.
It passes under the second prong of the Mayo test due to additional features that ensure the invention's claims are more than an abstract idea.
It has novelty and this invention is new; has not been done before. The process, teamed with various units that exist, are interchangeable, and readily available, is what forms the gestalt system dynamic and supports its variation and multiple use cases.

Part 4—Innovation and Government/Paver Readiness—Moderate Level

This short section illustrates high points that can be achieved with the invention in these areas.

Biotechnology

Inclusion with Biotechnology (Biotech) to pave the way for long term health and living including a focus on older age disease prevention or reversal in dementia by association with token based funding to support new discoveries and experimental approaches.

Government

One of the main goals that Centers for Medicare & Medicaid Services Innovation Center (CMMI) has established for the next decade is increasing access to care by improving affordability of care and reducing out-of-pocket healthcare spending.

CMMI will aim to lower the share of beneficiaries that delay or forego care due to cost by 2030. The centers will also measure whether all models have some method of lowering the costs of high-value care for beneficiaries. This invention achieves this.

CMMI identified five ways in which they want to improve affordability for beneficiaries:

First, the center seeks to lower program expenditures. This would have a ripple effect on beneficiaries' costs.

Second, CMMI plans to target and eliminate duplicative and wasteful care patterns. The centers noted that total cost of care models could be useful in this endeavor. This invention aids in this effort.

Third, the center plans to employ payment waivers that incentivize high-value care usage. Waivers could allow participants to offer home healthcare to prevent readmission after hospitalization or they could enable providers to lower out-of-pocket costs for primary care services. Tokenization of these waivers through our healthcare cryptosystem supports this goal.

Fourth, CMMI will leverage value-based insurance design (VBID) models to bring down costs and provide access to technologies−such as this invention and the innovative capabilities for beneficiaries to create their own health plans- and devices that can help beneficiaries manage their conditions.

Fifth, CMMI will employ financial incentives for providers to encourage them to pursue high-value care and cut down on low-value methods. Episodic payments could be key towards promoting efficiency. Collateralized “Flash Contracts” is one approach that achieves this objective.

To address health equity, new models will require demographic and social determinants of health data as appropriate; they will support safety net providers and will serve overlooked populations. The Healthcare Cryptosystem will identify gaps in health equity to confront.

Innovation

The invention creates new technology in hardware, firmware, materials, software, and methods. Much of our Mirror-database inspired by Metaverse concepts, are really derivatives of the real entity, much like financial Futures and Options contracts are derivatives of existing real commodities.

A key hardware approach is our use of Internet of Things (IoT) antenna technology is described in How to Build. We utilize a number of Proof of Work approaches including

“Proof of Network” in radio as introduces by the “Helium” Blockchain Token and process.

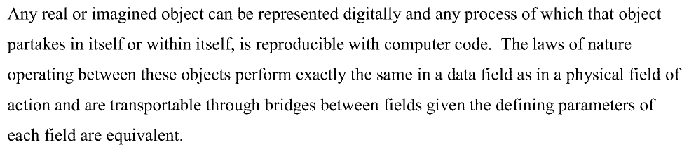

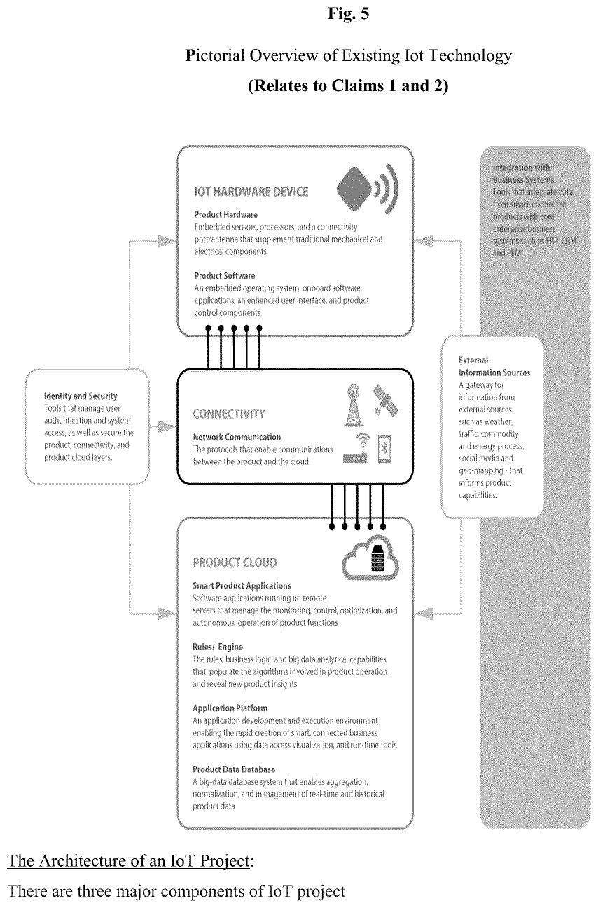

FIG. 5 presents a pictorial overview of existing lot technology and relates to claim 1. Division Segment 2

In this section, we address a number of “How to Build Steps” for most embodiments. But at first, we describe the detail steps in the Best Mode Embodiment discussed earlier, starting after the next 8 to 9 paragraphs.

There are a number of topic guide sections and instructions in this segment on how to make and build the preferred best use embodiment. Many of them employ unique approaches but they are all held together by a common design philosophy based on equivalency, natural law, technological bridging, and data representation between physical entities and programmatically coded pairs. By incorporating what today are multiple point software solutions within healthcare into a unitary system governed by interoperable software, standards and common rules, and not a centralized system, the invention removes error without sacrificing innovation. Point solutions introduce chaos and do not work in non-localized critical systems.

This is a detailed level segment. The reader with average skill sets normally found in those involved in associated various fields can build the embodiment.

Since we cover different disciplines in order to deliver the gestalt of the “Healthcare Cryptosystem”, the instructions may be obvious to some in one section, say with skills in creating semi-conductor antennae and integrated circuits or chips, but may need a different reader's skillset for the blockchain programming sections in order to understand interacting processes. Similarly, the person with blockchain programing skills may need to take a jump in understanding of the interacting applications utilized in biotechnology, neural networking, or analytics, to be comfortable in building the overall system from the foundational parts.

The complete deployment will occur in roll-out fashion and built and implemented in phases. Immediate advantage will be realized by government agencies involved with healthcare rulemaking and with any cost and utilization measurement related organizations and will then follow through to the general public affording significant benefits to patients and families and consumers at large.

We segregate “claimed” topics and clearly identify those that are unique to the invention. We identify subsequent topics where our claimed components interact with components that are not listed in our Claims Section of the invention application and already exist.

In this section of the Blockchain Healthcare Cryptosystem following the preferred embodiment, we address the foundational tolls for other embodiments. Therefore, specific steps are addressed that can be followed to build a working prototype of this Use Case and others not mentioned.

The actual invention is an industry encompassing mechanism and affects the workings of our current healthcare system's “AS IS” state and the transformation to a “TO BE” state via the invention, these instructions are illustrated in the Invention Drawings section and presents the use case now under discussion. The outcome of the fully executed invention has the capability to re-invent the industry of Healthcare Information Technology in many ways.

In this Preferred Embodiment Step-by-Step section we describe:
Introduction
How to build the token
Tokens
ERC20
IPFS
Unstoppable Domains
MetaMask
We then follow the detailed embodiment steps and cover:
Minting and Issuance
How to build a Smart contract
Emergent Capabilities
NESSI DEA Analytics with Dynamic Mapping
Avoidance of Threats to the Cryptosystem
Hardware, Firmware, Antennae, and IoT
Smart Materials
   Graphene semi-conductor material and 3d Printer paired coding
   Splicing Graphene on molecular level to biology DNA markers
Incorporation of X.25
GPS Mapping to Smart Contracts and IoT Antennae,

Then, high level discussion on the following invention high point components:
1) Graphene Antennae; Physical and Programmed, DNA/FFT-EEG Circuitry
2) Blockchains, Hash, Consensus, Tokens.
3) Emergent Smart Contracts (embedded in virtual antennae and as programs)
4) NESSI
5) Threat Recovery, Ransomware, Financial & Network Sustainability, Propagation

Step-by-Step Technology Build of Preferred Embodiment—Begins Here:

The Token Creation Process

As stated, the process of creating new tokens, or for example, BITCOIN, is the process of creating new blocks as defined by specific blockchain protocol. A blockchain is an immutable ledger where each individual block is cryptographically built from the blocks previous before it. Blockchain is therefore a grouping of “validated transactions” recorded on a given block and consensus mechanisms validate transactions to determine active chain and next chain. (See Hash Process description at the beginning of this section)

There are different types of tokens. Native tokens of “state of the art” public & permission-less Blockchains like Bitcoin or Ethereum, are part of the incentive scheme to encourage disparate groups of people or computer servers (miners) who do not know or trust each other to organize themselves around the purpose of a specific blockchain.

These blockchain-based cryptographic tokens enable “distributed Internet groups, network hubs, or ACO's” to emerge.

There are four basic types of blockchain implementations:
Public (permission-less)
Private (permissioned)
Federated, open only to federation or organizations
Hybrid

As RHIOTOKEN represents health units of value, these conceptual and practical descriptions are in order, especially in this embodiment building section. Our thesis contains these observations: Healthcare is a commodity and a necessity—the RHIOCOIN is not a security made only for trading; it has intrinsic utility value. Though RHIOCOIN and associated coins with the Healthcare Cryptosystem can be used for financial transactions, it is important to remember that payment for services or operations can many times be a mixture of fiat, commodity, and barter based processes. Commodities, Fiat, and Federal CBDC are all subject to fluctuation, going up and down in long term value and appreciation.

The nation cannot have its healthcare infrastructure that just so happens to exist in a capital based economy, become subject to the failure of any medium of exchange. This would include things such as price controls, deflation, credit and liquidity freezes, or bank failures to name a few. Also, the recent melt down of many cryptocurrencies in exchange based price, tells us that no one medium is sufficient for something that is the most critical function in our society.

The following is a description of exactly how to build a token for the Healthcare Cryptosystem, setting up an IPFS distributed network file system, and API or other interfacing mechanism process.

We can pictorially show the interrelationships of coin types and the positioning of RHIOCOIN as compared with others. See FIG. 9 which relates to claims 5 and 6.

Token Minting is currently performed under a Paragon service with Unstoppable Domains, whereas Token Issuance is planned utilizing Ravencoin. Ravencoin is a Token Issuance company. It creates new crypto assets by burning a certain amount of $RVN coins and providing a unique name for the tokens. Once created, special characteristics can be defined for the new asset such as number of tokens issued and fungibility. Fees are required to utilize the service. Ravencoin will work to get new token assets listed on various exchanges for additional costs. This process is akin to how an investment bank helps a private company issue stock and go public on a stock exchange.

Common terms:
Blockchain
   a publicly-accessible digital ledger used to store and transfer information without the need for a central authority. Blockchains are the core technology on which cryptocurrency protocols like Bitcoin and Ethereum are built.
Decentralized
   a system operated by a distributed, peer-to-peer network instead of a central figure or authority.
Minting
   the process of validating information, such as domain ownership, and registering that onto the blockchain.
NFT (Non-fungible token)
   a digital certificate of authenticity used to assign and verify ownership of a unique digital or physical asset. Unlike fungible tokens, NFTs are not interchangeable with one another. Unstoppable Domains are NFTs themselves, meaning there is complete ownership and control over them.
Wallet
   a software application or hardware device used to store the private keys to blockchain assets, such as cryptocurrencies and NFTs. Unlike a traditional wallet, a blockchain wallet does not actually store the coins or tokens themselves. Instead, it stores the private key that proves ownership of a given digital asset. (e.g. Metamask, Coinbase Wallet, Ledger, Trezor)
Wallet Address
   also known as a public key, this is an alphanumeric code that serves as the address for a blockchain wallet, similar to a bank account number. Unstoppable Domains replaces these addresses with readable domain names. Digital assets can be sent to wallets via the public key or domain name, but only the owner can access a wallet's contents by using the corresponding private key.
Web3
   the next iteration of the web. It leverages blockchain technology, open-source applications, and the decentralization of data and information. Web3 aims to remove control of the web from central companies, returning ownership of data and content to its users. Also referred to as the “decentralized web” and the “read-write-trust web.”

IFPS and MetaMask Wallet

A brief overview of the InterPlanetary File System is in order. The InterPlanetary File System is a protocol and peer-to-peer network tor storing and sharing data in a distributed tile system, IPFS uses content-addressing to uniquely identify each linked with no central authority.

IPFS is a decentralized network where peers are connected and share files, similar to BitTorrent. The fundamental principle is that unlike the traditional web, where files are served based on their location, in IPFS files are served based on their content

Building a Blockchain—

How to build a Proof of Stake (PoS) Blockchain in “GO”
“Go” is a Blockchain programming language for creating Ethereum Tokens (ERC20+Verified)
This token will be a standard ERC20 token; This means it sets a fixed amount to be created.

Determine:
The Token's Name
The Token's Symbol
The Token's Decimal Places
The Number of Tokens in Circulation

### E.g.

Name: RHIOCOIN
Symbol: See Coin Logo's
Decimal Places: 0
Amount of Tokens in Circulation: 100,000

Coding the Contract: Generic program can be found using a Google search or ChatGBT AI and filling in the fields with the values given above.

End Detail Best Embodiment Technology Build—See Flowchart FIG. 10 for Use Case.

Below begins our component sections:

Component Summary Description Topic 1—

Graphene Material integrated with Semi-conductor based Antenna and DNA FFT-EEG Circuitry
Physical Antenna—EEG-FFT
Physical Antenna—DNA

Summary Listing of Topic 1 Steps will not be included at the end of this topic as steps are embedded in below narrative

Component Make-up, Features, and Attributes—Topic 1—

We utilize semi-conductor chip antennae that securely and uniquely represent a person's identity. We pair two antennae, one physical and one virtual within the Healthcare Cryptosystem. Our unique person markers are a combination of encoded DNA sequencing applied to graphene material and a second layer of unique identification derived from brainwave signatures that are specific to each person. These chips are circuits that generate their own power (See work of University of Arkansas physicists Pradeep Kumar and Phil Thibado who are experimenting with energy harvesting chips, “Fluctuation-induced current from freestanding graphene, Physical Review E, 2019). Graphene and circuitry share a symbiotic relationship. (Even diamond electronics, as another offshoot, or allotrope of Carbon, have applicability to chip and circuit creation utilizing high pressure and high temperature radiation tolerance).

Normal current in a resistor causes it to heat up, but Brownian current does not. It has been found that at room temperature, the thermal motion of graphene does induce an alternating current in a circuit. Why this is important to the invention is described further below. In short, the electrical catalyst in both the actual live waves and cloned virtual frequency waves we create in our mimicked antennae pair, allows us to use Inverse Fast Fourier Transform algorithmic mathematics to simulate a wave with frequency, on an ongoing basis, for the virtual antenna in our electronic Metaverse-like electronic atmosphere. We devise a synchronous polling mechanism in the real world to match our virtual world waves. This creates real time dynamic person matching that is not anchored to static representation and therefore is very difficult to hack.

EEG
Brainwave with DNA identification followed by DNA exists in our graphene and virtual antennae.
Brainwaves are collected via collection devices that combine neurofeedback leads or by acquiring a person's waves through the process of “Brain Mapping” or qEEG.

Brainwaves are measured in terms of electrical activity over time and the relative dominance of different types of waves can be identified. These brainwave groupings are many but for our purposes we limit the dominance classifications to the 6 most common. These waves are 1) Gamma measured at a frequency of approximately 35 to 80 Hz (Hertz being cycles per second with regular periodic variation); 2) Beta measured at a frequency of 12 to 35 Hz; 3) Alpha measured at a frequency of 8 to 12 Hz; 4) Theta measured at a frequency of 4 to 8 Hz; 5) Delta measured at a frequency of approximately 0.5 to 4 Hz; and 6) SMR or “Symmetrical Motor Rhythm” which is a blend of waves in a single moment identifying the state of the person as well as the unique pattern of a biological brain signature.

Brain wave mapping from the person via electrodes and stored through a variety of instruments such as those by companies such as DataQ and MATLAB that have the added capability of running wave patterns and frequencies through a mathematical algorithm known as a Fourier Transform (FT). This Transform is the system approach we utilize to mimic the physical brainwave signature from the physical antenna associated with the person to the paired virtual antenna. The FT decomposes functions depending on space or time into functions depending on spatial frequency or temporal frequency. The Fourier inversion theorem allows many types of functions to be mimicked from its Fourier Transform. The Discrete Fourier Transform (DFT) converts a finite sequence of equally spaced samples of a function into a same-length sequence of the Discrete-Time Fourier Transform (DTFT). Since analogous transforms over any finite field, such as brainwaves, can be applied, the inverse DFT is the same as the DFT but with the exponent having an opposite sign or simply, a 1/N factor for relatively easy adaptability.

Quantum sensors and brain scans for eeg encoding, gps accuracy and sensitivity, electrical currents within the brain generate magnetic fields can be collected via devices capable of magnetoencephalography (MEG) at room temperatures. MEG are akin to atom interferometers.

(leee spectrum, “A Quantum of Sensing—Atomic Scale Bolsters New Sensor Boom.” June 2022)

DNA

DNA synthesis is done today and, over the last 5 to 10 years, has progressed to nanoscale circuit boards such as with “Im perium” microchip technology or company projects such as Agilent, Twist, and Gen9.

All DNA synthesis has two basic steps:
Make short fragments of DNA into Oligonucleotides and apply enzymes to bind them;
Synthesize them into microarrays such as on a piece of glass the size of microscope slide.

A middle ground layer is needed and it is satisfied by holes inside of nanowells of a silicon wafer. Alternatively, another optional approach that could be applied would be a chassis-independent recombinase-assisted, genome engineering (CRAGE) tool. One is made by Lawrence Berkeley National Laboratory. Costs are lower than ten cents per DNA letter and less than 20 days turnaround time

From a software perspective, interacting components, bridging through hardware Oracles, creates a “Blockchain Based Healthcare Entity.” It utilizes technological “GPS-like” virtual coded pulsing (polling, inspired by unit defined “ticks”—(a borrowed concept from financial “Futures” market price movements)) antenna within the coded environment (Frequency can be represented and mimicked in code using integral based Calculus mapping). These software processes map to active ledgers with a unique networked and dynamic “Overlay” relational database.

Units are programmed and populated in “near-real-time” through emergent smart contracts. These contacts (or active software receptacle packets) use distributed code in the Blockchains that are triggered by “Utility Tokens (such as RHIOCOIN's),” while interacting with other token types that each utilize other Blockchains. (Celsius Exchange and Cardano have similar multi-blockchain goals). These Smart Contracts and the secondary tokens follow emergent processes as smart contracts themselves are then created. Using a combination of NFT and encrypted non-NFT tokens, the pairing of a patient with all treatments, history, evidence, places, relationships, etc., are mapped and tracked and joined dynamically within the database and as clones of the physical person with attributes of the physical world. The invention utilizes physical and virtual IoT Antennae and semi-conductor molecular level spliced material.

From a hardware and firmware semi-conductor prospective, the software is bridged through graphene, built into antenna technology with attributes that the invention encompasses. The evolution of IoT technology is possible because of advances in designing and manufacturing of electronic components enabling robust connectivity in integrated technologies.

Interacting tokens may be Ethereum “ETH” or Polkadot “DOT” or Cardano “ADA”, or similar technology that are readily available to the general public with GIT-Hub Open Sources such as Helium network tokens, and the IOTX platform. Our primary RHIOCOIN Token is based on the Ethereum ERC-20 and ERC-721 standard. We also utilize many other tokens, blockchains, multi-coins such as Oxijim and Coinmonks, and exchanges such as Celsius, already mentioned.

Non-Fungible Tokens (NFT) represent the patient themselves anonymously and any other secure and anonymous information. Any other information about the patient or the patient's current or potential care are inherited and joined in the database on a “need to see” basis at the proper time and by consented providers or processes. Protected data includes HIPAA designated legal health record sets based on AHIMA definitions and CFR42 that further protects sensitive patient data. The system invention also works in “Shard” (Not “Shared”) versions of the same Blockchain node and supports multi-native tokens in a single Blockchain. (See Qi Zhou, QuarkChain, Aug. 22, 2018)

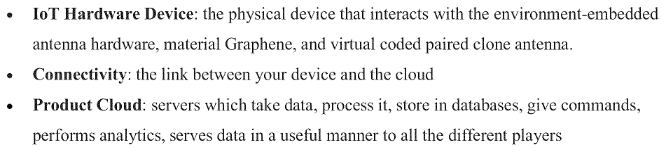

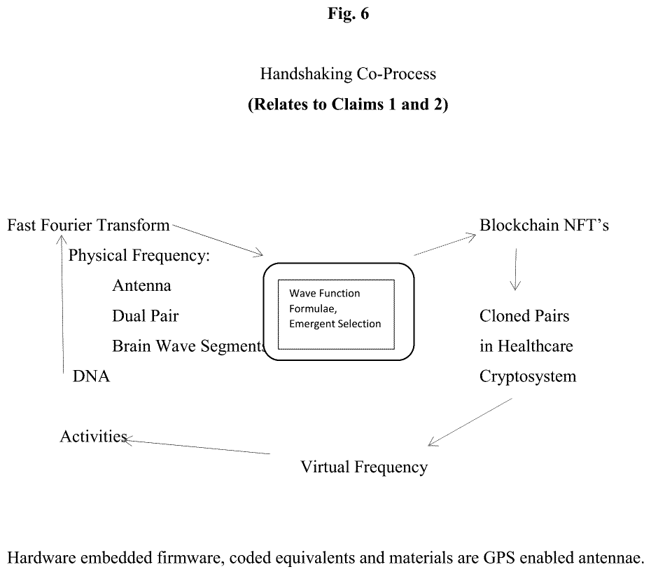

When the concepts described above interact with the hardware and embedded firmware next described, a handshaking “co-process” occurs. See FIG. 6 for a high level pictorial representation of this handshaking: FIG. 6 relates to claim 2 and by extension, claim 1 as well.

Monolayer graphene exhibits properties owing to unique, regular arrangements of atoms. Graphene is usually modified for specific applications. Some of the critical parameters of the material for use by the invention are in the formation of bonds, electric band structure, and the number and stacking order of the layers.

Graphene film consisting of only a few layers was once employed to fabricate transistors due to its strong and bipolar electric field effect. The materials became building blocks in nanotechnology and are used for this invention due to electronic band structure. Graphene is an allotrope meaning it is one of a form in which an element can exist, as in this case, graphene, charcoal, and diamonds are all allotropes of carbon.

The electrical properties of graphene can be exploited in more direct ways due to the two-dimensional honeycomb lattice structure. For example it is utilized following a standard antenna blueprint which can also be further sub-divided by monitoring a current through a narrow graphene nanostructure. This then contains a nanopore through which a DNA molecule translocates for example, and the material follows a second blueprint for specific DNA sequencing. We incorporate this approach to form the specific NFT that matches a specific patient with no chance of error or mis-identification.

By combining semiconductors acting as antenna and interspersed with molecular DNA and EEG code, we create links to so-called avatars (smart contracts) between the person representation in the Metaverse and the real person representation with their attributes. These are the tags and the tags are key as portals to the Metaverse. The attributes are collected by emergent and dynamic smart contracts created randomly by “mutation similar” natural processes and awaiting pairing when conditions are met or by being deleted after a parameter (usually time) is exceeded. When conditions are met, the joining to the networked “Overlay” database occurs.

In addition, it has recently been discovered that the natural thermal motion of graphene can be incorporated into a chip to provide low-voltage power for small devices such as a credit, debit, or any kind of portable card that this invention can utilize as an Oracle gateway from the physical world to the mirror database (Metaverse) of the healthcare cryptosystem.

As stated, any material existing in physical form or in idea, can be coded into a superstructure of programs and networks. The laws of nature acting between such objects and any processes of that object that partakes in itself or within itself, is therefore reproducible. This is much like the Metaverse where unique moments in time represent the electronic atmosphere from the perspective of user.

The laws of physics and nature exist everywhere and are consistent in all aspects of life. These laws are applicable not only to science but to other systems such as economics, statistics, biology, the stock market, and even politics and human behavior. These observations play out over the long term as well as the short term in observations of the fluctuations in the timeline for the emergent smart contracts.

To expand on this concept further, the reader is referred to a photon analogy process not fully comprehended by the general public, yet a significant mechanic in the physics of energy and light. Common belief accepts that light can exist both as particles call photons and as a wave function, and that light is bounded by a maximum speed limit of approximately 186,000 miles per second or 299,792,458 meters per second. Delving deeper, this duality in nature and speed is a derivative of mathematics.

In a photon “movement” mechanical model, how light “travels” when the photon has no mass is explained. Experiments have found that one part of the process is less intuitive that what formulae show. In reality, a photon, or a packet of energy, is produced when a neighboring electron of an atom strikes the orbital of another atom's electron in the outer shell. As the electron that was struck moves back to its original position in the orbital, it creates a second packet of energy (photon again) which appears in the visible light spectrum due to its frequency. This new photon appears to move on to the next closest atom with an electron shell that can be dislocated and the process repeats. So, what is known as the speed of light is a sequence of electrons bouncing back and forth in orbitals and giving off visible light energy in photons, and appears in observation as “light moving.”

How does this fit into the invention and within this embodiment building section? The answer is that by knowing how to dissect how things work in nature, we can simulate these activities in code. This includes the building of virtual antenna on virtual material with molecular structures to pair with physical counterpart antenna with the assistance of major material management engineering companies in aerospace, CHIP design, and with assistance from ChatGBT AI. It allows us to mimic frequency in a coded bandwidth that matches electromagnetic frequency in the paired antenna in physical form for GPS effects in a distributed programmed network. It allows coding DNA sequences in virtual coded chips and antenna smart contracts from real DNA partial genome sequence models using hashed outputs. We gain this inheritance aspect for the physical antenna graphene material and through biological samples through interlacing with semi-conductor boards.

This also allows identification and insures that we have the right person symbolized in the antennae; the smart contract; the NFT tokens and non-NFT tokens of peripheral units or concepts; and other entities. They may be physical or representations and make up the total health of the person or cohort. It also allows us the capability to use distinct hash copies from the Metaverse equivalent environment in order to gather health insurance payment claims and diagnosis data from government systems such as the “Blue Button” project for Medicare and Veterans, and to glean data from commercial payers and hospital/practice records now allowable under “Price Transparency” regulations. This is all with done under HIPAA compliancy and patient consent and with safeguards built in to avoid fraud by someone losing or having their physical antenna card stolen.

We code equivalency between the physical and software pairs existing in the cryptosystem using advanced 3D print software technology to mimic hardware and frequency. Microstructures created by a 3D printer give us tools that work at the Nano level. In addition, integration of different graphene nanostructures with PDMS (polydimethylsiloxane) in composite-based compact wearable devices detect physiological states of wearers by sensing electrochemical signals and EEG waves as described above. These can then be matched to our virtual coded paired antennae in our Metaverse, much like “Digital Clothes”. Ultimaker's S3 models are example devices. We create on CAD and slice with settings. We then create prototypes with many mechanical or electronic properties that have material compatibility.

Since these paired physical and coded antenna structures can both emit signals (real world frequency and coded equivalent frequency in the Metaverse database) we take GPS capability to locate and map the real antenna and do the same within the cryptosystem coded antennae using a coded GPS-like process in the frequency mechanics and we map within the cryptosystem as well. This then leads to normalization and the ability to correct disorder and bad or incorrect information and transmit back to physical antennae via 2-way encrypted pathways.

Our antenna based identification approach is both physical and virtual. Healthcare identification and regulations against sharing or misusing Protected Health Information (PHI) and Personally Identifiable Information (PII) without consent, is highly protected and a critical aspect of the Healthcare Cryptosystem. There are points within the system that are both in the coded form and in the physical world where a balance is needed for the invention to deliver yet not compromise the safety and the privacy of people and entities within it.

From outside of the system, we use identification of the person or entity owning and using our graphene based antenna and identification cards. These cards not only have GPS capability but also acts as gateways into the mirror Metaverse database via Oracle capabilities.

Our algorithms also employ Steganography processes where invisible communication, such as hiding text messages in audio or video files, is employed to thwart hacking and to promote secure use by an ACO.

To insure as much certainty as possible that the owner of the physical antenna card is the correct person, we combine multiple identification scores in a weighted approach following SICPA (Societe Industrielle et Commerciale de Produits Alimentaires) and we combine those identification parameters with actual physical “Touch DNA” which creates unique low cost DNA profile matches based on relatively small biological samples. Of the 3 billion base pairs of genomes (A—Adenine, T—Thymine, G—guanine, and C—cytosine) which reside in the 23 pairs of chromosomes within the nucleus of our cells, only about 0.1% is enough to provide an accurate identifiable profile of a person. This is because 99.9% of that code is exactly the same in every human. And of this amount, there is an even more manageable tally of 21,000 protein-coding genes.

With DNA coding alone, identifiable person data could be compromised and misused by outside entities. By adding the unique person based FFT, with always changing parameters to each antenna and keeping in constant check with physical antenna synchronizing (Ack/NaC bisynchronous simulated polling), the data approaches a non-alterable state.

The aforementioned SICPA is a private Swiss company formed in 1927. It is used as an advisory organization to public governments, central banks, high security industries, and others in defining official formal foundational physical identity parameters. Its methodologies address the inherent lack of immunity to counterfeiting, alteration, unsecured identification, lack of traceability, and the need for authentication of a digital identity.

To build secure and identifiable features into our Antenna that is to be held by a participant of our system, we combine parameters of the SICPA (Financial, Health, e-Commerce, Labor, Mobility, and Government Records) based on a numerical assigned list of documentation with multiple levels of precision and with parameters returned from “Touch DNA” lab results represented in data format. We combine these numbers into a unique hash and validate it using a combination of computer generated validation processes of the source data and the consent and agreement of the person. This is much like a Blockchain Miner would use to validate a block in a ledger. In our case, “Mining for Healthcare,” is a process performed by multiple layers including the participation of the individual legal owner of their own health record.

In addition to identification data, the physical antenna is built so that over time, it inherits the longitudinal health record of the person as the card is used for other activities such as payment for service or as an identification card. It, and the location of historical health services, is also locatable via real and paired virtual GPS. Antenna-like tags in the software labels and indexes peripherals as exposed over time

After passing through the entry portal of the physical Oracle through to the electronic atmosphere of our mirror database, a coded matched virtual antenna is electronically paired with the incoming code. This antenna is part of a smart contract that works through the mirror database, correcting inconsistencies in data and matching with other tokens, both new and existing, and it only finds other emergently created tokens where a need exists. For example a missing piece of history, evidence of disease or family DNA risk, a need to perform a prior authorization process, etc. are just a few cases. When a match is found that requires an action, this combination of events is viewed as a “Trigger”, and the process kicks of a new smart contract of stored code to perform the action and return the results.

How the emergent tokens are found within the mirror database is via active coded mimics of antennae attached to the tokens as sub-routines and follows actively coded GPS virtual system with coded equivalent frequency-like code. It uses bisynchronous polling related network protocols sending payloads within the cryptosystem based on X.25 packet switching. Antennae and equivalent GPS transponders in the virtual database are then coded, as previously stated, using advanced 3D Printer technology coding. Mutations of non-functional, or non-needed, evolved smart contracts and tokens are deleted following parameter rules.

Based on programmed parameters, these processes regularly pass through other Oracles that transmit updated normalized data back to the real world and populate the physical graphene antennae of the end user. Over time, all inconsistent data that has been normalized and corrected in the mirror database, is applied to the real world to adjust the inaccurate data on non-interoperable health care software systems.

The schematics for the antenna and semi-conductors will be presented to a short list of vendors in the fields of Antenna Technology, “Smart Materials” Producers, and 3D

Advanced Printing companies. The building of prototype electronic chip board schematics based on the invention specifications will follow after publication is scheduled. The steps are:
Draw Logic Flow
Select Board Designer
Create Circuit Board Flowchart

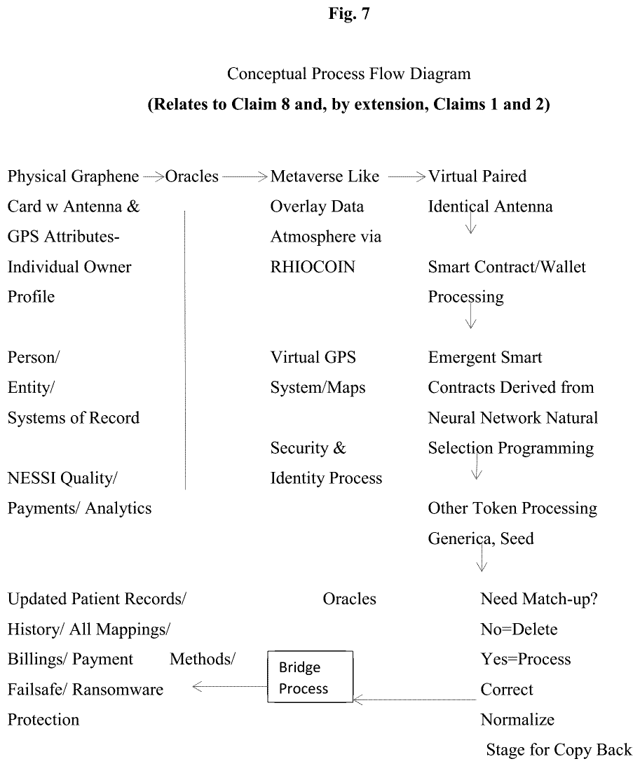

See FIG. 7 for high level “Process Schematic Flow Overview.” This is a conceptual process diagram and relates to claim 7 and, by extension, claims 1 and 2.

Our Antennae Oracles send data to and from the outside world. An example of an Oracle in Blockchain is the sending of daily temperature, to a blockchain such as Ethereum. Smart contracts on blockchains use the data to make decisions about what action to perform. A blockchain Oracle can use a third-party service that connects smart contracts with the outside world, primarily to feed information in from the world, and also the reverse.

The defining quality of Blockchains like Ethereum is that they are able to run smart contracts. Once programmed, smart contracts are fully controlled by the blockchain; no entity needs to be trusted to execute the rules, and no middle service or entity can prevent the transaction from taking place, assuming the conditions for the smart contract are met.

It should be noted, if an Oracle is a data feed that is run by a 3 rd party entity, a potential problem of trusting the data source can lead to data integrity issues. The owner of an Oracle's data feed could post inaccurate data by accident or in order to sway the smart contract. Alternatively, some may hack the data feed. Smart contracts that are not dependent on Oracles don't have this problem. Our approach is for Oracle computers to use Trusted Execution Environments (TEEs), special areas of hardware with extra security, making them difficult or impossible to tamper with. Bar coded materials, antennae, or equivalent mimicked bar code computer code are other approaches.

Regulatory advances in Medical Hardware—SBOM and EAAR are also key components. Another progressive approach positively affecting hardware in healthcare is the Software Bill of Materials or SBOM. The SBOM provides a list of all software components in a given device, enabling transparency by allowing device manufacturers, buyers, and operators to identify and mitigate vulnerabilities and manage medical device security more efficiently. An executive order signed in 2021 on improving cybersecurity in concert with the US Department of Commerce, along with the national telecommunications and information administration (NTIA), is publishing guidance outlining the minimum elements for a software bill of materials. Analogous to an ingredients list on food packaging, an SBOM is a list of all included software components and provides a transparency mechanism towards security in the software and product supply chains. When used in the medical arena, and even in Metaverse based virtual equipment, it is a start to standardizing hardware and interoperating software applications with a secure approach that the invention incorporates into its mapping processes.

Also regulated Bio-Medical associations such as EAAR (European Association of Authorized Representatives), address Medical Devices from the prospective of privacy, security issues MDR and IVDR, and legal issues arising out of implementation. National competent authorities involved with notified bodies address regulatory compliance; conformity of compliance requirements; scope-borderlines-classifications; and safety and performance requirements.

Component Summary Description Topic 2—

Blockchains, Hash, Consensus, Tokens

General Description—Specific Steps Follow

Hash Process, Block Creation, and SHA256 (Secure Hash Algorithm)

When someone requests a transaction using blockchain technology, it is broadcast to a peer-to-peer network consisting of computers known as nodes. These employ algorithms to validate the transaction and the user's status. Once the transaction has been verified, it is combined with other transactions to create a new block of data for the ledger.

In the case of a traditional bitcoin blockchain for example, some items are consistent across other blockchains. The Bitcoin hash number is created from random number generators such as “pseudorandom number generators “PRNG's”, and “CSPRNG/CPRNG”, cryptographic secure pseudo random number generators using key generation (digital signatures), SALTS—random data appended to inputs/passwords and fed into hash functions. The algorithm then randomly chooses the next validator/node in blockchain.

This process is dependent on concatenation, one-way functions, a numeric seed, and the basic rules imbedded in SHA256 (SHA=Secure Hash Algorithm), and reconciled through Merkel Trees with their roots. Parameters of a hash include the Height (timestamp), NONCE (a “number used only once”) as an integer number in the 256 range and added to the hash. This distributed ledger technology (DLT) thereby creates immutable records where smart contracts that are blockchain specific execute a set of defined rules valid on only that blockchain. For this all to work, fault-tolerant consensus mechanisms must exist. In our case, the Healthcare Cryptosystem uses its own blockchain and consensus protocol and also uses interoperating external blockchains where consensus is inherited and reconciles by the distributed network nodes.

Change of any piece of the Blockchain is not possible, as everyone connected to the network will easily see the changes and refute the version of the blockchain under a “Proof of Work” or “Proof of Stake” approach or similar. PoW is known as an early consensus mechanism or consensus protocol. There are many others and will be touched upon further. Some include Proof-of-Authority (What RHIOCOIN uses; an advancement to the Proof-of-Stake ERC20 Ethereum based Token), Proof-of-Storage (as used in FileCoin described later), and Proof-of-Coverage, which is a consensus mechanism to validate networks used in the Helium Blockchain, which we use to distribute our physical antennae to the public.

Metadata going through the hashing algorithm is encrypted based on SHA256 as the statistical mathematics needs to be guessed by miners (unless using PoS described below). Text within the hash may or may not be encrypted, but can be loaded by utilizing various hexadecimal mathematical operations such as “Zero and Add Packed.” The text (patient medical record) can be inserted as Hexadecimal into purposed fake addresses in the hash and can be done by products such as Python or simply a skilled programmer. A preferred method of encryption on the text field which we use is the same process used to encrypt smart cryptographic images in NFT creation processes. These are then the readable fields open for validation by patient and provider with the proper private keys to view it.

The general method that is used to make blockchains work as described follows these steps:
A grouping(s) of “validated transactions” are recorded in a given block.
A newly-formed block is “connected” to the preceding block forming a “chain.”
Consensus mechanisms are used to validate “transactions” (to be inserted in new blocks in the chain and to determine the “active chain” when delay-induced) such as RH IOTOKEN abeyance.
As new blocks are “seasoned” (when block rewards are released), an irreversible set of blocks is created in an ever-increasing chain.
We now go through the significant components of A) Consensus Protocols; B) External Blockchain Interactivity with RHIOTOKEN—“The Opportunistic Blockchain Ecosystem” and; C) Token Creation.

Consensus Mechanisms and Protocols

As stated, Consensus mechanisms are used to validate “transactions” and insert new blocks in a chain. Proof-of-Stake (PoS), is an up and coming popular consensus mechanism to replace Proof-of-Work which is a highly wasteful approach to validate blocks by human effort, computing effort, and electricity/energy use.

PoW, PoS, Proof-of-Authority (an algorithm, used in variation by the Suncoast RHIO and RHIOTOKEN, is based on identity as a stake such as with VeChain), Proof-of-space/storage, proof-of elapsed-time is an Intel based lottery consensus approach to equalize chances of validation are all up and coming consensus mechanisms.

Heium's PoC, Proof of Coverage, is used where mining is based on validating transactions called the bespoke mechanism (Hotspots on the network are randomly and automatically assigned with Proof-of-Coverage tests to complete) and use compatible Hotspots. In the case of Helium, HoneyBadger BFT protocol is used.

An example of the processes and the constraints of a PoS approach are: When a validator is chosen and creates a new block, it is awarded a “Block award” Validator then passes most of block award to staking participants as staking rewards. Validators also receive transaction or gas fees for computational expense on the node such as smart contract execution.
Staked crypto must be staked for a prescribed amount of time.
Validators pay interest to parties contributing to total stake.
Slashing is penalty mechanism for bad behavior or not living up to terms.
Scaling uses Sharding in Etherium 2.0 for splitting database to spread the load primarily for transaction validation.
NFT can forcible assert conditions based on ACO.

In practice, and similar to PoW but without the high concentration of workers and electricity and without wasted effort once a block is solved, PoS takes the statistical approach that a block will be solved by someone. So the randomizing software as assigns a “winning miner” and when that validator is chosen and creates a new block, it is awarded the “block reward.” These validators then pass most of the block reward to staking participants who contributed to the miners’ odds of being chosen by being awarded the native crypto currency in most cases. PoS, the software, and governing mechanisms establish how algorithms function, and the order of the protocol steps, can be thought of as a “Proof-of-Authority” approach and is how the Healthcare Cryptosystem evolves its own “Proof-of-Patient/Provider/Pharmacy” under a PoA approach. This subset protocol is known as “PoP.”

As we describe later in the Healthcare Cryptosystem blockchain consensus protocol, “PoP”, or this Proof-of-patient/provider/pharmacy/payer,” is done after the first level of miner validation using a PoS Metadata approach (traditional “hash guessing”). Following that, a second challenge moves the mining activity to validation by text confirmation of the patient and the provider (or additionally, the pharmacy and/or payer in some cases). This is done after or in concert with the traditional “Hash Guessing” miners' activity of validation.

Abeyance routines are employed by the software to hold the solved segments before a reward is generated when the other segments complete validation. So, segments are throttled, or slowed down in a latency program, and released by certain triggers, such as confirmation of the patients' records. Holding rewards to metadata miner patients and providers as miners for too long until the full block is validated would not be an incentive for all parties to partake in the mining effort. A timing mechanism is also therefore used that releases portions of rewards to those miners that completed their steps earlier.

PoP is the consensus mechanism for RHIOTOKEN as described here:

In the healthcare cryptosystem, there are a number of dynamic processes that are currently processing or, set for validation a short time after transaction submission. RHIOTOKEN and its currency, RHIOCOIN (RHC), utilize the operations of established outside blockchains as well as its own PoP. Primarily, RHIOTOKEN blockchain interacts with: UNIT; Cardano; Helium; Polkadot; BitCarbon; FileCoin; Bitcoin; Ethereum; and soon Fedcoin, in addition to other lesser used Tokens and as determined by the stage of operations.

The overall mechanism for validation of RHIOTOKEN blocks falls under variations of “Proof-of-Authority” as stated. In this system, all patients and providers are now incentivized to correct, keep, and check medical documentation and records. There is no longer a need for an unsecured national health identification number, security is increased and viable, and risk is reduced.

Where does the value of the RHIOCOIN come from? When purchase, it is backed by valuable commodities like diamonds or gold; the work of the validation process by all miners: the utility to providers, hospitals, and researchers and more so to have a validated ledger with validated patient record fields that can be independently transmitted to others while remaining consistent and up-to-date.

It is also backed by the investment return in offering toolkits and toolkit bundles to support innovation and health technology startups; and dynamic patient defined “pay-as-you-go” health-plans. When backed by Fiat form, the backing takes the legal stance offered by “promissory estoppel” in that, as a debt instrument, the Fiat itself is a contract with terms that requires that the “purchasing power” of the unit of fiat, when used on this blockchain, must retain that purchasing power whenever redeemed in the future in order to avoid inflation causing policies of 3rd parties. In terms of “staking”, our system approach is different as it is minimally related to mining process rewards and more related to collateral investing in an upwards moving market. The need for healthcare and its value cannot go down.

These PoA validation sub-mechanisms (A, B, C, D, E below) are determined by the parties involved (who are also the miners and who receive RHIOCOIN's as shared rewards that are formulae determined within our software, for block completion) and the scenario of the operation. They are:
A Primary PoP Grouping—Most Common:
   PoP1—Proof of Patient (does the patient agree with the data (EHR) on themselves)
   PoP2—Proof of Provider (does the provider agree data is accurate and should be validated)
   PoP3—Proof of Pharmacy (do all pharmacies agree with medications reported including OTC)
B Hospital and Facility Grouping:
   PoP4—Proof of Patient Durable Medical Equipment (DME) based on “Bill of Goods”
   PoP5—Proof of Patient Facility (if relevant), Hospital, Practice, ACO, etc., IPPS and MA Plans
C Health Information Management (HIM) and Billing Group/Company Grouping:
   PoP6—Proof of Patient Quality/Accuracy of Record (ICD Codes, Documentation Match, MIPS)
   PoP7—Proof of Provider Compliance Governance
D Smart Contract/ACO Grouping:
   PoP8—Proof of Patient/Provider COIN Revenue Basket Validation: Commodity, Fiat, Crypto, CBDC (New blocks are not created until previous block basket value reconciliation is validated)

E Insurance Company, Group, Government Payer Agency

PoP9—Proof of Payer, consistency with X.12 EDI EOB patient bills and provider claims.

These 5 PoP variations are grid and software array determined. Not all are appropriate to every operation and not all miners are the same, though there may be overlap. Smart contract software determines which PoP variations apply.

There is also a double and sometimes triple, accuracy consensus that insures all validations in the cryptosystem are synchronized to the physical layer. This is known as “Proof-of-Oracle” (PoO), and is carried out automatically by the software and the antenna synchronized firmware that acts as the miner under the auspices of governance.

Endpoint Medical Record validation result reporting to patients and providers takes place through these Oracles via antenna cards, websites, Explanation of Benefits and other EDI reports, and optional output available to patients via Federal Government Health and Human Services Division (HHS) under CMS (Center for Medicare and Medicaid Services) Agency's “Blue Button Project” Reporting.

A question that comes up fairly often and has been addressed in other sections of this invention bears repeating. With all of the hacking and criminally orientated technologists looking for ways to breach anything of value, especially healthcare, the most valuable data of all, how does the system avoid hijacking of its own software and smart contracts? The approach used is one of triple checking across networks, ecosystems, and environments: 1) Consensus, the system utilizes three layers of mining and cooperative miners that are always being synced by software and firmware antenna; 2) Personal checks and alarms to those with graphene cards are alerted to changes or out of sync conditions and to reconciliation processes in an ever present distributed network. Alarms are also logged by ACO's at certain levels; 3) FFT Frequency Distribution in real time matched to a current state of bonding between the holders EEG wave output and encoded antenna algorithms.

These exist in both the hardware and in the parallel network of the system or what is referred to as the Meta-environment. These items are difficult to hack as even if a coder with criminal intent were successful over all of these boundaries, by the time they could act on the information, it would have already changed.

The Opportunistic Blockchain Ecosystem

Healthcare Cryptosystem's approach to Leveraging and Optimizing External Blockchains

As stated, the Healthcare Cryptosystem utilizes its own software as well as interoperating with existing blockchain technologies and other blockchains in any layer. Our approach supports a cohesive distributed network control mechanism as opposed to a centralized one. Software Smart Contracts along with their hardware antenna paired oracles mitigate the counter party arbitration processes with added security and redundant layers that are kept in check synchronously. The adoption of this approach allows us to position our approach as a “Token-as-a-Service” or, alternatively, a “Token-as-a-Server” solution.

There are a number of tokens, technologies, and blockchains that we interface with. Our architecture also allows the creation of new smart contracts based on evolutionary parameters of natural selection via neuro programming. As old tokens and blockchains become obsolete and new blockchains enter the metasystem, the invention accepts, adapts, and uses these new entries. Our smart contract capability that evolves these new tokens, NFT's, and blockchains, and result in some of these new innovative entities. This is described fully in its own topic in this section.

This interoperability is for both creating our own new tokens as well as our software inheritance of existing tokens and blockchains. Some of the inner workings to two of our key 3 rd party blockchains, Helium and UNIT, will be used to distribute our antennae (produced with radio manufacturers and “materials” firms) and will be moderately described upon in this section. Others are mentioned and described at a high level in order to keep the overall mechanism in context.

We refer to this cooperative circle of interoperable blockchains as the “Opportunistic Blockchain Ecosystem.” Shared security among blockchains is performed under the “Unified Consensus Aggregation” Polkadot project by Shafkupe from Saint Petersburg, Russia under Yuriy Vin. The initial primary blockchains and/or Crypto assets in our “Opportunistic Blockchain Ecosystem” are:
UNIT
Helium HNT
Cardano
Polkadot
Substrate
Bitcarbon
Bitcoin
Ethereum
Filecoin
Fedcoin (when available, also called FedNow)

These descriptions are only moderately described in the context of the workings of each of these blockchains, and does not go into detail. That information is available on the respective websites and white papers of each of them; and are not components of the invention except for the interoperability code that is created to link to them

FIG. 8 is a pictorial representation of the “Opportunistic Blockchain Ecosystem.” It is related to claim 8.

Unit

The UNIT Network is one of the key systems that the healthcare cryptosystem interfaces with. Bei, ng a participant of this network allows RHIOTOKEN and our users (including our own Smart Contract generating software and our antenna software oracles) to create and mint tokens; create governing organizations to manage new nodes outside of the initial Suncoast RHIO; and wrap and unwrap tokens from any layer so as to take advantage of existing blockchains such as Ethereum, Polkadot, Cardano, HNT, and Bitcoin. This leads to operational advantages by making the Healthcare Cryptosystem's first use case populated and fast-tracked. UNIT is used by our natural selection token/smart contract creation process.

In addition, UNIT has native protocols with its token called by the same name, “UNIT.” It has methods that support financial operations. We can stake and wrap tokens to the UNIT Treasury enabling assets such as commodity backed cryptocurrencies to perform as collateral. These backed crypto assets include BitCarbon (Diamonds), and Quints (Goldbacks) from the United Precious Metals Exchange in Alpine, Utah (UPMA.org). We then perform staking or lending via decentralized network mechanisms. In this case, the system becomes the custodian of the assets and the smart contracts execute our code. Consensus takes place using a Proof of Authority (PoA) model similar to Parity Systems.

Unit Network is a layer 1 blockchain that was built using the Substrate framework by Parity Technologies/Web3 Foundation. It is free for businesses, projects, social communities, and individuals to create crypto-tokens, also known as ‘community’ or ‘social’ tokens. This is represents a new approach to crowdfunding and shared ownership. It sets the foundation for innovation in healthcare technology. It enables us to offer collections of tools and toolkits to new and upcoming businesses to use. Use cases lead to innovations and startups when we supply the toolkits. A cost saving is that the very steep and complicated healthcare industry's technology learning curve is greatly reduced. No longer do developers have to spend years understanding one of the most convoluted industries in the country or to attempt to think like physicians. And having these toolkits supports “Just-in-Time” inventory and streamlines the supply chain with these incubated startups.

As supporters and participants of the SEDSI (Southeast Decision Sciences Institute), the builders and participant of the healthcare cryptosystem, we submit ideas and accept new ideas in the International Journal of Innovation and Learning (IJIL).

Unit supports distributed security and immutability as components supporting our systems accelerated access to healthcare entity domains and financial services with mitigation of counter-party risk. Every token created on Unit Network is able to open a decentralized exchange pool known as DEX. The protocol used is called an AMM (Automated Market Maker). This is where financial interactions occur with the RHC (RHIOCOIN), the currency and reward mechanism of the Healthcare Cryptosystem.

Wrapping and Unwrapping

Blockchains like Bitcoin and Ethereum have different protocols and functionalities. Due to fundamental differences in their algorithms, they do not interoperate.

Blockchains such as Polkadot overcome interoperability issues. However, it became necessary to find solutions and allow communication between early networks like Bitcoin and Ethereum. This is how wrapped tokens were first created.

Wrapped crypto tokens, when introduced were used for financial operations. They are cryptocurrencies pegged to the value of another original crypto or assets like gold, stocks, shares, and real estate and work on Decentralized Financial (DeFi) platforms.

The original asset is ‘wrapped’ into a digital vault and a newly minted token is created to transact on other platforms. Wrapped tokens allow non-native assets to be used on non-native blockchains. They build bridges between networks and implement interoperability. The Healthcare Cryptosystem utilizes this wrapping for both financial transactions and utility healthcare transactions with other tokens in the network.

The current working process for wrapped tokens that is used in DeFi begins with a request of a merchant or other initiating user to hold the token in custody and create a new wrapped equivalent. By a similar process, when the wrapped token needs to be converted back into the original asset or a coin like Bitcoin, the initiator requests the custodian to release the token from the “Treasury” reserves.

In the standard financial wrapping process, a custodian is required, however with the UNIT processes, these activities are automated in smart contract code.

The RHIOTOKEN and it currency, RHIOCOIN, abbreviated “RHC,” utilizes it’ own blockchain consensus mechanism (PoP) as described and is based on “PoP” variations, “Proof of Patient,” “Proof of Provider,” and “Proof of Pharmacy.”

Additionally the Healthcare Cryptosystem RHIOTOKEN inherits consensus from the blockchains' it interacts with to validate those blocks. The UNIT mechanism under the UNIT process combines authority with both smart contract code. Validation is also doubly achieved by mirror networks and reconciliation by patient/provider/pharmacy miners and “bridge oracles” between the physical and the Meta-environment.

Helium
The Internet of Things (IoT) Hardware Antenna Distribution
One of the prime mechanisms to physically disseminate Healthcare Cryptosystem
Combines with IPFS, Metamask, Wallets, & Bridges to the Crypto Metaverse Blockchains

Understanding Helium and the relationship of this blockchain network to IoT, a starting point would be to look at past efforts such as MIOTA, a popular “block-less” platform that was used to connect devices with IoT. Sometimes called IOTA, it is the crypto coin of MIOTA, a distributed ledger designed to record and execute transactions between machines and devices within the Internet of Things (IoT) ecosystem. There are hundreds of cryptocurrencies addressing a wide range of use cases, however, there are relatively only a few that embrace and support the Internet of Things technologically.

Helium is the chosen blockchain network that performs this required function for the Healthcare Cryptosystem. Helium is described as a blockchain-based network for IoT devices that uses nodes as Hotspots (defined by them) and that connects wireless devices to the network. The native token of Helium is HNT and, in order to entice miners to validate using the Helium consensus mechanism, HNT both serves the network and also serves as a payout when Hotspots transfer connection data across the network. As the need for the Internet of Things grows with the development of technology, Helium is relevant for the invention's network reach and antennae distribution.

Helium technology enables communication between devices and also sends data across network nodes. Nodes that compose the network are the “Hotspots” in Helium parlance and not to be confused with the public use of the word. The Hotspots provide public network coverage and rely on LoRaWAN. LoRaWAN is a media access control layer protocol that has a cloud component to which Helium can connect.

Helium is a significant LoRaWAN network with over 25,000 Hotspots that act as nodes on the network. These Hotspots combine LoRaWAN capability with mining devices based on blockchain technology.

As with other blockchains including our own RHIOTOKEN, there is either a community or a ACO that governs, or has involved people personal collateral in the system. As of 2022, the Helium Community approved HIP 51 which enables more wireless networks to join the system to complement IoT. Helium's working mechanisms supports RHIOTOKEN antennae distribution of the Healthcare Cryptosystem.

Native WiFi already has support for IoT devices. However, support for every wireless device brings up privacy concerns. Helium uses a decentralized architecture and consensus mechanism that gives the network 200 times greater coverage as compared with WiFi connections only.

Helium “Proof of Coverage” consensus mechanism is based on the HoneyBadger Byzantine Fault Tolerance protocol. It allows network nodes to reach consensus when connection rates vary.

Additionally, aside from using the native HNT token, Helium employs another token, which cannot be exchanged, and which can be used to pay transaction fees. These are known as Data Credits.

Hotspots are balanced against the HNT which is burned to generate Data Credits in a Burn-and-Mint Equilibrium token model. The inflation rate decreases over time, ensuring that the total supply never exceeds 223 million.

Software wallets provide options. Online wallets or web wallets are accessible from multiple devices using a web browser. This is in line with the RHIOCOIN that is recommended for example, with the MetaMask Wallet on an IPFS platform.

Tokens are smart contracts and the RHIOTOKEN and antennae in the cryptosystem are as well. The Helium token HNT is an ERC20 token in the Ethereum Mainnet and is implemented as ERC20 with address 0x4cb9eb266cdd03f3f4a8c64bb990613f41993bf5.

Helium is a critical component to the invention as a major pathway into the hardware world. This is how connection is made to consumer credit/debit cards build on the invention's graphene chip devices described in another topic in this section. Helium is used for distributing, maintaining, and upgrading antennae and for growing the network to reach all consumers of healthcare.

Further in-depth information on the workings of Helium is available in the invention appendix.

Filenet and FILECOIN

Filenet (FIL) from Protocol Labs (not to be confused with IBM) and FILECOIN is a decentralized storage network that turns cloud storage into an algorithmic environment. In the case of mining, power is proportional to active storage. Storage can be rented out and users can select replication parameters on the IPFS network. It uses “proof of storage” schemes and “proof-of-replication”, and “proof-of-retrievability” via validated health information.

Users can associate programs to their transactions such as with the OpenEHR systems EHR (Electronic Health Record) and is workable within the Ethereum blockchain. Bridges are used as tools that aim to connect different blockchains with Filenet.

Cardano

Cardano and the BeefChain Model

The Healthcare Cryptosystem utilizes an approach that keeps track of all entries and entities in its ledger list. One large blockchain that performs this is Cardano. Cardano blockchain has many applications and smart contracts. A successful implementation that performs this function for the meat industry is known euphemistically as Beefchain.

Our implementation of the Cardano Blockchain for healthcare ledger tracing, data entry, and record of changes follows the approach used by Cardano with Beefchain.

Traceability is a priority in the healthcare industry. There are many unique entities that make up the field and each one requires a secure record. There are thousands of these unique entities and examples include materials; hospitals; medications; patients themselves; their diagnosis and procedure history; places of service and time service was rendered.

Some of these entities are coded as dynamic) fields that are unique and can be represented in blockchains as NFT's or Non-fungible-tokens. Others are reusable, such as EKG machines, and are therefore fungible. However, many times, even these fungible entities, at a high level, may have sub domain categories with non-fungible parameters such as representing patients who used the equipment; where and when; history of maintenance records or problem are examples.

See sample listing of the various healthcare nomenclatures that need to be addressed in FIG. 3, Lists A through E.

in terms of risk, of all industries, Healthcare Risk tops the list. Once a health risk is discovered, providers, companies, and governments alike struggle with the repercussions. An immutable ledger is required. The healthcare cryptosystem uses Cardano Blockchain technology to allow users to trace their health services and products. We intend to partner with IOHK, the technology firm behind the Cardano public blockchain, IOHK helps in the technological aspects of their blockchain tracing endeavors by supporting them in using the Atala Trace solution,

Devices, patients, and services—physical or in virtual electronic form, get tagged with RFD tags that are either real or virtual. Another used of the Helium network described above would be to distribute RFID on the antennae acting as oracles,

Using these tags, entities have a unique identity and can be tracked as they move through the healthcare chain. Consumers can access this identity using a OR code which once scanned, will take them to the website and the data source from which theft record was produced or last updated.

Component Summary Description Topic 3—

Emergent Smart Contracts (both embedded in virtual antennae and as programs) Summary Listing of Topic 3 Steps will not be included at the end of this topic as steps are embedded in below narrative

Component Make-up, Features, and Attributes—Topic 3—

A smart contract in this context is a computer program or a transaction protocol which is intended to automatically execute, control, or document legally relevant events and actions according to the terms of a contract or an agreement. How smart contracts govern and how tokens get allocated are definition of conditions of transfer.

We utilize concepts of vectors as qualities with a direction, sometimes thought of as particle. When overlaid with a plane say in the shape of a 2 dimensional xy graphic axis, we can create directions, for example the x axis, and we call these basis vectors in a vector space, where descriptions can be made by adding attributes such as speed of movement of the vector, existing time expended in contract life, location as related to the basis vectors, or whether the vector of discussion is a “smart contract” moving and existing in two parallel vector spaces; one in the physical world and one overlaid in the metaphorical “Mirror Database.” A “Timelock” is a smart contract that delays function calls of another smart contract until after a predetermined amount of time has passed.

Emergent smart contracts are a hybrid mix encompassing simple smart contracts, coded antenna pairs, and neural network programming. Based on the 1984 work of Douglas Lenat and Cyc, historically cheap computers gave access to a grid approach and enough power to run neural networks with many layers of artificial neurons and led to systems which became known as deep neural networks and an approach known as deep learning. Geoffrey Hinton at University of Toronto applied a principal called “back-propagation” to make neural networks learn from their mistakes.

In “AI, Neural Networks, and Deep Learning” by John McCarthy and the “Symbolists” in 1956 and “expert systems” in the 1980's followed by the “connectionists” of neural networks with one pioneer being Frank Rosenblatt using 400 light sensors are important pioneers in the field. The chip maker Nvidia known for GPU's used in gaming systems and blockchain “Proof of Work” applications, performs sophisticated kinds of shading and geometric transformations, and can be tricked into doing other tasks such as Al layered processing and active programmed Neuron programs in Neural networks. This allows training of neural networks with the CUDA platform example. In neural network learning systems (deep learning from the many layers of processing going on at once) must embrace “catastrophic forgetting” in order to continue learning. This is what gives us the 2 sided AI related Neural models; one being operational with 3 layers—Input, Hidden layers, and output layer; and the second being the training and learning and runs in reverse of—Ouput layer; to the various hidden layers; and finally the input layer.

The emergent contract process works as follows: Smart contracts are programs stored on a blockchain that run when predetermined conditions are met. They typically are used to automate the execution of an agreement so that all participants can be immediately certain of the outcome without any intermediary's involvement or time loss.

An emergent smart contract is programmed to follow the laws of evolution and natural selection in order to create random mutations (self-replicating contracts that may have a use and those that do not). The idea being that the Healthcare Cryptosystem cannot have every solution possible within its workings, nor can it adjust for unpredictable future events or needs. It would need very high maintenance and labor costs to keep it functional if it were even at all possible. Again, we borrow from nature. We look at what are the terms of evolution and how can we program these terms. There are many programmed natural selection model engines and programs done by neural network scientists that are created to show how biological life evolves even when applied to programs (or “creatures” as the specialists say).

David R. Miller, a neural network specialist and programmer has stated the following, “whatever reproduces, reproduces; whatever doesn't, doesn't.” Substituting the word replication for reproduction, we have our basis to start.

In biology, genomes get passed on and can be shown in simulated network brains. Using simple selection criteria, neural networks and genetic inheritance simulators use complexity and organization over time. We create data structure scripts and we copy those data structures. In the human evolutionary tree, there are 3 billion genes. We use much less for our contracts; anywhere from 10 to 20. We program in mutation capability into our own software simulator. ACGT letters in DNA is replaced in our approach with short genomes, similar to “meta” fields. We allow for parameters of: 1) Size; 2) Number of grid points; 3) Number of dimensions; 4) Degree and number of grid points of movement or steps; 5) Population of fields; 6) Realm of Healthcare Domain from our “Mappings”; and other attributes designated in our simulated brain neuron map.

The conditions necessary for evolution and natural selection are the following:
Something that self-replicates;
A Blueprint (such as Genome stored as DNA or RNA, as storage descriptors);
Inheritance capability—inherited blueprint such as from 2 parents to a child;
Occasional mutations (parameters in simulator by random chance in order to create unique contracts);
Selection method such as in nature—eg. Which contract gets to reproduce and which ones do not in order to survive (and replicate) within intervals of lifespan.

We apply the disciplines of:

NEAT—Neuro Evolution of Augmenting Topologies, an evolutionary algorithm that creates artificial neural networks with each genome containing 2 sets of genes that describe how to build the network; contains node genes, each of which specifies a single neuron, or in our case, a part of our “Bundled Hub” incorporated from the invention's “Medical Hub Exchange.”)

Self-propagating and evolving smart contracts and new tokens are best for creating, adding, and modifying new programs for unanticipated and overlooked conditions and for healthcare entity attributes. As in a “grid computing” approach, contracts naturally attract to where to be and how to survive if meant to be; much like how cars in rush hour all know when to get on the road at once to be a the critical location of work.

In our dimensions/attribute choices for the contract parameter switches, we use the following at a minimum: A) Timestamping as a composite of space and time for contract uniqueness; B) Activity logging; C) Payer Designation (To support dynamically created user health plans on the fly); D) Age of contract and if used or not picked up by other use cases naturally over X time; E) Brane Memories.

We use what we call “Brane Memories” to create our unique composites and to insure longevity of experience existence for other future contracts. This is the memory overlay function. We introduced the concept of an “Overlay” dimension to our database correction and Metaverse normalization process in the architecture section of the invention detailed description. The same approach is applicable to emergent smart contract avatar/antennae in order to address the preservation of memory over contract generations. As the contract entities uniqueness is a number existing only once in all time, the eternity of the number is not addressed by the other numbers around it (hashes) but only within it. This is a function of DNA sequencing and FFT wave composition.

The term “Brane” is borrowed from string theory multi-dimensional propositions that state the some items (strings) in an entity existence are short lived and most likely 2 dimensional, where is there is a coexistent overall string that brides the gap over space and time and is most likely defined in 3 dimensions. In string theory, this is how gravity is defined as a force.

Component Summary Description Topic 4—

NESSI DEA Analytics with Dynamic Mapping including (Nested Envelopment Statistical System Interface)

Component Make-up, Features, and Attributes—Topic 4—

The NESSI system is built utilizing API's and Python Flask programming languages. One of the calculation modules in the Smart Contract that is used to present provider scores contains business rules and sub routines. One is programmed with “Data Envelopment” and “Frontier” non-parametric comparison programs. These compare entities that optimize a define end goal such as may be reflected in the real legal contract between the provider or provider group and the payer in order to set reimbursement and comparative rate data.

Each aspect is score-able, dynamic, and weighted and statistically determined in machine to machine processes kept secure by algorithmically and encrypted data flows in a living model.

This process determines counter-party governed triggers governed by HIPAA Business Associate Agreements that calculate and execute the “Inventory Mapping.”

DEA is a method of using Nested Data Envelopment Analysis of quality measure groups to devise a risk shared saving index (NESSI) usable by medical providers and health insurance payers and other funding sources in Accountable Care Organization negotiations/Ongoing contract performance benchmarking.

NESSI can also be used as a model in health information technology businesses that focus on quality measures and reporting during the formation stage to determine viability of a new business, eClinic, or Franchise.

We utilize DEA (Data Envelopment Analysis) to perform calculations on cohorts of accepted quality measures. We add physician satisfaction and benchmarking.

As benchmarking is the process of comparing business processes and performance metrics to industry best practices and can be used to compare from other industries, an index can be created where similar processes exist. This approach is used to help explain success of the firms that do well and others that do not. Much like capital markets, where tracking value as based on price of commodities, in a futures' options or index entity, the fair value approaches a zero dollar difference as the contract moves closer and closer to the settlement date. (See published paper by author) However, variations occur as volatility appears higher, the further out from the contract date we examine.

We use NESSI to constantly measure this change so that funders of contracts to healthcare providers will know when an investment or a funding commitment is on or off target, and when intervention needs to occur. This can also be applied to the actual measure of care as well as the dollar amount of the contract attributed to the care unit negotiated.

Benchmarking is often treated as a continuous process in which organizations continually seek to improve their practices.

As two of the most common approaches to identify strong and weak performing units (regression analysis and DEA), we use these to quantify units of subjective quality with quantitative units. DEA estimates the cost level an efficient entity, such as a firm, that should be able to achieve in particular market.

DEA can be used to reward entities/operators whose costs are near the efficient frontier with additional profits whereas regression analysis only estimates what the average entity or firm, should be able to achieve.

Provider satisfaction is key here, as a provider will not perform at his or her best, when a subjective target, such as healthcare outcome, and patient satisfaction is involved.

We choose measures that can be gleaned from accepted AMA measure codes and measure groups such as CMS MIPS, or others identified in such groupings as NCQA HEDIS, Joint Commission, or Patient Centered Medical Home (PCMH).

Our cohorts are buckets of measures which are then viewed in the neighborhood frontier. Hence, if we view that the operations are occurring in groups of measures instead of just individual variables, we are nesting the groups and treating them as one for the purpose of generating an index.

A Payer oriented index of 1.0 might be reflected as a lower number from a provider standpoint as would a provider optimized index be lower from the standpoint of the payer. However, this is the number that is the starting point of risk determination and negotiation and is universal and standardized in use.

The uniqueness of adding cohorts based on benchmarking and provider satisfaction measures into the mix of nested quality bundles make this index a superior indicator for all parties.

Indices thus generated are variable for different optimizations and can reflect change. By using our specific computer generating programs, we collect and act upon data that has been DEA processed and optimized for at least 3 categories of interested parties: Providers of medical services, payers of medical services, funders’/investors of ACO groups and franchises. As time moves on, we find a 4^{th} interested party, the consumer, with a say in new ACO formation or expansion of an ACO's services and reach, but at this point, the consumer is not enough of an organized entity to benefit from our approach. This is where a governing process enters the cryptosystem.

When ACO's are formed, there are at least two parties involved, each with different approaches as to which quality measures should be optimized to have the highest return. For example, providers will choose measures that are most closely aligned to their specialty, in which population they have most success, and where financial outcomes are secure and process driven. A payer or funder of a new ACO or of an ACO in review, might instead concentrate on measures that are outcome driven, have higher or lower risk populations based on their appetite for return, and wish to see funding returned early in the cycle rather than the safer and more predictable annuitized path.

CMS restrictions such as minimum beneficiary target population and strict geographic boundary definitions, make some ACO's more challenging to form and operate at agreed upon targets. Investors may find that by taking newer model ACO's and instead investing their funds into local, private, and specialty based groups, partial returns of investment income streams can be a annuity that guarantees a return of the original portion of the investment early in the funding cycle. It allows for modifications of measure indices as an ongoing feedback loop by as small as a quarter instead of a year or two. Thus, a tranche approach to risk and return can act as a cushion to the investment, giving funders a tighter control on performance through periodic reviews of the index instead of by direct management control of doctor or hospital practices that might be rejected by the providers. The ACO governance index introduces a team approach to managing health and financial returns instead of adversarial.

The use of this index” opens the door to standardized measurement of financial return mixed with clinical achievement to positively support good clinical practice. Introduction of consumer choice through the use of publically available and trusted websites that are actually used by consumers, will drive consumers to choose ACO's that meet their needs; will marry providers with affinities of a specialty to those that need this care; and will optimize financial flow to all parties while cushioning investors with a small offsetting annuity return. All this happens while the bigger prize of outcome optimization is proactively managed by the involved risk parties on each side acting as teams. They have less confrontation due to the approach of continuous feedback and continuous quality and financial optimization improvement.

It is possible that as ACO's or “ACO—Like” organizations progress, and subjective clinical outcomes move closer to quantifiable clinical outcomes in lockstep with optimized quantifiable returns, using approaches like this, being more understood by the financial community. They can become ETF's of their own, go public, be purchased, and attract the best customer to their services at the best price. Of course, all of this tempered by public policy controls and avoiding monopolization of groups while adhering to anti-kickback controls.

Component Summary Description Topic 5—

Sustainability: Threat Recovery, Ransomware, Financial & Network Sustainability and Propagation

Component Make-up, Features, and Attributes—Topic 5—Though this component presents as a mix of topics, they are all interrelated in that they form the basis of key processes by which the invention sustains itself in financial and operational processes. Protection against healthcare threat and network availability is embedded in the mechanical and electronic workings of the system. Additionally, financial viability addresses the definition of currency and the earning and spending of funds to healthcare providers. We also address both automatic network propagation and its close cousins of financial viability, stability and growth.

It is important to note that the inclusion of all methods and technologies in this section are not claimed for the invention. The need to address already existing approaches that we plan to incorporate into the invention is required in the narrative. In fact, in this section, the only mention of our claims is in the network topologies using our antennas, and in the financial section where we mention our “Dynamic Build your own Health Plan” capability. The order of this section presentation is:
Threats, Recovery, and Ransomware;
Physical Network Sustainability, Propagation and Topologies;
Financial Sustainability and Self Growth;
Additional Funding Sources.

Threats, Recovery, and Ransomware—System Failure and recovery vis-a-vis Natural Failure or Attack

Ransomware

66% of healthcare organizations experienced a ransomware event in 2021. This is up from 34% in 2020 and a 94% increase over a year. The healthcare industry experienced a 66% rate of attack and experienced the highest increase in volume of cyberattacks at 69%.

Operationally 94% of the attacks impacted the ability to operate and averaged one full week to recover from an attack. 77% of those affected put faith in approaches that empirically found, do not really prevent attacks.

The cryptosystem invention takes secure patient and other secure files in an encrypted state. They can be held encrypted by the domain of distributed networked servers without the capability of any party or program except for the owners with their private keys to transfer back and decrypt. In addition, based on banking protocols, networks are segregated by access layers. One of the more common approaches is the “Red, Green, and Blue” network where human access is only allowed on the Red layer, network interoperable API and processes on the Green, and only system activities on the Blue. The nature of encryption in concert with blockchain hashing and immutable ledger inherently is a disincentive for hacking.

Threat by Labor Shortage

13 million is the global shortage of nurses predicted by 230 without sufficient recruitment and retention.

79% of medical groups that said prior authorization requirements increased from 2021.

57% of U.S. small-business owners expect economic conditions to worsen next year.

Our “Toolkits” introduced in topic 2, allows moderately knowledgeable individuals to used functionally pre-defined toolkit bundles of common groupings of healthcare tasks so that they can be proactive and productive while learning.

Physical Network and Grid Threats

There are many variations of potential threat to the system. We will address the most common here starting with electricity. Causes of local or global failure due to electricity can take many forms. They may be caused by simple local failures, regional and national grid malfunctions, hostile actions by nation adversaries, or major class X electrical magnetic pulse (EMP) from solar flares to name a few. Recovery of electrical power failures and/or loss of internet accessibility is achieved through varied means in the packaging of the invention's fail-proof processes.

Examples of these items include a combination of computer to computer programs and associated hardware (we based encryption wallets) that enhance performance and store records in offsite devices. Also, cryptographic cold storage wallets can include solar power back up and alternative “fail-over” to other internet providers, land-based or orbital. This insures that work done by the provider is always registered on a mirror copy of a synchronized network DNS server “Official Record” on a secondary live database that remains untouched by human data entry or edits. It is validated by Blockchain participants unknown to each other around the world, and are addressed and kept secure, unalterable, and accurate.

The use of technology via satellite internet servers keeps the crucial operations of the healthcare cryptosystem working regardless of surface chaos. By combining the IPFS filing system with networks such as Tesla SpaceX Starlink for example, we can see how the goal of seamless continuity would occur. In our Starlink example, we have historical data. A crucial role was played in the recovery of the 169 islands of Polynesia Tonga when a volcano erupted and resulted in a tsunami that destroyed undersea communication cables. The Kingdom was able to continue its communications via the SpaceX system.

Other private companies such as SpaceChain's Constellation offerings, Blockstream, Cryptosat, and Hughes are rapidly entering the market space with orbiting satellites offering blockchain validation, multi-signature wallets, and verifiable time-delay gaps for adjustment and audit. We additionally address the potential threat to the “unstoppable” demands of decentralized networks with blockchain validator nodes in space.

This means that even if nodes fail, are compromised, or shut down—or the internet is somehow turned off, a verifiable copy of the blockchain will persist in space, adding to the “immutability” and censorship-resistant attributes of the technology

Also, as previously described, our graphene physical antenna cards that pair with their network virtual clones, utilize the Brownian Motion electrical self-generation aspect of the graphene and card design.

Network Sustainability—Network Topologies

Thanks to standard setting leadership from such organization as the IEEE and ANSI, there exists a host of different yet compatible topology methods to use and sustain the network. For example; short wave, peer to peer functionality with Distributed Name Services server nodes, X.25 software packet switching with hardware and virtual PADS, and radio transmission from some of our support collateral tokens such as those from DiamondStandard.co radio nodes (described later) are just a few.

Wakoma's Nimble, an open-source, portable, wireless mesh network system comprised of form-factor networking hardware housed in weather proof casing will be used. Wireless mesh networks connect large areas by using radio nodes and are resilient. Wireless network on card antenna is designed in our schematics. Healthcare may well be the killer application that propels wireless mesh mainstream.

Mesh performs by using non-line-of-sight (NLOS) wireless and does not require installation by technicians thereby keeping costs low. NLOS defines its own smart antennas which we incorporate with ours. Advanced modulation techniques offer a potential collection of mesh architecture on regional and local networks known as “Air Hoods.” When used, in sync with “AirHeads,” which can be radio nodes based on diamond radio transmitter coins which then can also act as collateral.

Advanced modulation techniques like orthogonal frequency-division multiplexing (OFDM) use the available radio spectrum thereby maximizing the number of bits per second that they transmit per hertz of spectrum bandwidth.

This approach allows exploitation of multipath distortion. Radio communication complications caused by multipath distortion, where signals bounce off various objects and reach their intended target out of phase because they traveled more than one path, are addressed with old school packet switching approaches. With smart antenna array topologies, network systems such as the X.25 standard rebuilds the unit of information at the destination points.

Distortion is then turned into an asset. This applies equally to simple virtual circuits. By analyzing complex signals received from each subscriber during routine handshaking, the same array decomposes it into a number of simpler signals, each characterized by its strength, direction, and time of arrival, which are all components. These can be correlated to the data that is represented in the payload of the person or healthcare entity within the cryptosystem. As stated earlier, healthcare is the application for the network that may result in unitary standardized network adoption in mass.

Financial Network Sustainability and Funding Pathways

In this part we address the healthcare currency and financial sustainability of the Healthcare Cryptosystem—what is it, how it's created and earned, and how it is spent?

The basic foundation, the processes, and the requirements are expanded here at high level.

Revenue Emergence—

In a world that seeks sound money that acts in the role of units of trusted intermediates of exchange between any parties in a transaction, it is hard to imagine a more critical exchange than that one between a patient and a provider. Medical physicians hold a special and unique place in our society; somewhere between talented professionals and extended family members. These talented people hold one of the highest and most important places in our society and one that touches each of us on a personal level.

Within this kindred relationship, individual providers also need proper compensation for their efforts and in a currency that holds value and purchasing power. Helping a person or family avoid hardship and pain or recover from disease, has a present value primarily determined at the point of the episode, diagnosis, procedure, or plan of care. This currency unit must be created and be representative of the value rendered as closely as possible (Many outcomes of medical provider intervention are, of course, priceless). What that currency is should be of their choosing, and not dilute in value or purchasing power over time.

We look at currency and currency equivalents. We also look to collateral pegs to items of publically desired and accepted values such as commodities, fiat currency, labor value based units reflecting current and future value in the form of savings, newly created or existing cryptocurrency coinage, unitary values determined by locality and community, barter, and mixtures of the above. We will also consider a peg of the RHIOCOIN as a floating peg measured against the IMF market basket ADR, the CBDC Fedcoin, and the act and rewards of different mining approaches where miners' value can be described as the ombudsman of the system.

Starting with labor, the following parameters are considered: What is it we individually possess in specific abundance that is in demand and lacking by our neighbors, counterparties, and merchants of desired goods and services?

What do we have that represents consistently produce-able “labor units” and what are the consistently produce-able items that can be traded for those labor units? What is it that we can do daily or over any defined units of time in order to offer value to others needing that labor as we define it?

Units of currency value are determined by the accumulated effort of the labor to produce and held for a future date. Borrowing this future labor instead of saving it, disrupts the purchasing power and unnaturally rewards those that had no stake in the creation of value. Value can be represented in physical commodities of guarantee but should not be borrowed from in the form of debt.

Two strong examples of physical mediums are Gold and Diamonds. In our cryptosystem, Gold is represented by Goldback dollars issued by United Precious Metals Association in Utah which our RHIOCOINS are purchased in reserve. These commodities can also be directly purchased, held, and used in the cryptosystem and as directly by participants. Goldbacks can be easily redeemed into fiat dollars or UPMA Alpine Gold cryptocurrency tokens known as Quints. Users become members of UPMA at no charge.

Gold, though historically and relatively stable in value and purchasing power, still displays short term volatility against world capital markets and fiat currencies. Something in the way of a more stable entity therefore needs to act as collateral and this choice has been made in the cryptosystem for using Diamonds or Diamond tokens known as Bitcarbon, soon to be issued by Diamond Standard (www.diamondstandard.Co).

In addition, for the purpose of maintaining stability between Dollars, Gold, Goldbacks, Bitcarbon, and other cryptocurrencies, the system allows a repurchase arrangement similar to that used by the US Federal Reserve overnight window. In that system, banks can receive cash in return for collateral (or collateral for cash as in the reverse repo facility). In the case of the healthcare cryptosystem, we use the same concept.

One party using the repo facility desiring RHIOCOIN of our example ACO can be advanced by distribution of units of the Healthcare Cryptosystem (or simply US digital US dollars in debit format or CBDC including US Fed Coins, JPMorgan LINK, Circle's issued USDC Cash stablecoin, etc.) in return for pledging the hard asset of a standardized value Diamond or its Bitcarbon crypto token equivalent. This process is facilitated by the “UNIT Network Blockchain.” This collateral must be repurchased from the facility owner using Goldbacks in a set period of time. On the other end of the transaction, when users wish to have the Diamond standard collateral instead of coin, say in the case of excessive liquidity, a reverse repo operation occurs via a non-central Web 3 distributed authority. Whatever funds are acceptable by a provider in payment for services or by a hospital, or even a payer, goes into the “ACO like” sponsored Health Savings Account (HSA or MSA) of the participant. Also, in this way, the participant takes advantage of the invention's “Dynamic Personal Health Plan Builder” introduced in the detailed description of the invention.

We chose Diamond Standard Company as the venture has found a way to use diamonds as uncorrelated and under-allocated assets. As the diamond is a hard asset, is precious, is rare, and as noted below, is now able to be standardized giving the coin the advantage of being fungible, it fits our requirement. The diamond token Bitcarbon is also both a hard asset and a cryptocurrency and is tradable by a custodian with access to the coin key via radio wave transmitters. We use this approach in both our interactions with “UNIT Network” blockchain and “Helium Network” blockchain.

Being what is called a “market maker”, the Diamond Standard company bids on diamonds and purchases equivalent statistical samples using technology. This technology avows the determination of a public “Diamond Standard.” In this case, each commodity coin contains an optimized set of natural diamonds from a sample that add up to equivalent geological scarcity of carat weight, color, and clarity.

Financial Sustainability and Self Growth

There are a number of key approaches the invention will use to kick off the initial localized project utilizing Suncoast RHIO with the RHIOCOIN utility token and cryptocurrency capability set with pegs to commodities and labor. This is not an exhaustive list but represent the key initial approaches.
Additional ACO Member Organization Onboarding Activities with Franchise Potential;
Possible ICO Launch for 50% of RHIOCOIN;
Mining Activities Utilizing Unique and Various Efforts;
Collateralized leasing of Goldbacks vis-a-vis Cryptocurrencies, Commodities, and Fiat (FedCoin);
Component Launches via Kickstarter/lndiegogo selling hardware compatible products;
Mining credit rewards given for individuals using and distributing antenna cards;
Proof of Coverage Modeled on Helium HNT;
Rewards Expanding Confirmed Network Points for Expansion as Modeled by Helium Network HNT Token;
Rewards for Using Compatible Tokens via Cardano or Parachains;
ACO Based New Participant Stimulus;
Investment Clubs;
Government Grants & Awards Supporting PCORI (Patient Centered Outcomes Research Institute);
Usage Charges to Related Companies Onboarding as hubs;

As the example ACO progresses, more will evolve (and may have their own coins but not required) and governance will be local just as healthcare is primarily local.

We allow Goldback assets purchased through Suncoast RHIO from UPMA and held by participants to lease it back to us for an annual lease percentage equivalent to interest on a savings account.

The Healthcare Cryptosystem's accepted currency of Goldbacks for the initial Suncoast RHIO, RHIOCOIN, allows users to make payments to medical providers or payers. As a funding mechanism, we wish to entice RHIOCOIN holders' with added benefits. A second level of collateral exists by having Goldbacks and RHIOCOINS to be backed to Diamond Standard coins. Simply backing volatile coins of our own for lending backed by other volatile coins is unwise. This is why our crypto assets are commodity backed or backed by US Government Fiat, dollars or CBDC (Central Bank Digital Currency).

Additional Funding Sources
Distribute antenna cards, short wave, currency of goldilocks, nodes of diamond standard Bitcarbon coins;
Indiegogo Product Sales;
VC Investment;
Use of Antenna Cards for Mining Credit;
Network IOX Mining;
Diamond Standard Bitcarbon Repository held by ACO;
Leased Radio Token Collateral (HNT).

AHIP Health Care Triangle grants based on the plan and project achievement of:
Lower Cost of Care;
Access (having health insurance);
Quality;
Other Grant Proposal Topics;
Payer Closed, Open, Provider, and Defined Networks Innovation Grants Based on:
Evidence Based Practice and Improvements in Care;
Non-care Delivery Components of Tracking;
Substantial, meaningful, and measurable improvements in care.

What is claimed is:

1. A healthcare blockchain network apparatus comprising firmware, software, and hardware and characterized by easy-to-use by public consumer held distributed IoT (Internet of Things) SIM, eSIM, and/or credit/debit card based antennae graphene DNA and EEG FFT encoded chip cards, potentially in organization with other carbon allotrope materials, and acting within and as mobile cell phone based blockchain oracles of said apparatus; and with synced paired metaverse-like modeled Mirror-Base Web3 environment platform of parallel coded antennae; a distributed network; smart blockchain contracts; and unique PoP consensus protocol, so as to immutably record, track, sync, correct, secure, and reconcile disparate computer based health record physical components using VM, VT, Virtual Floating Desktops to health payment systems, and transactions of payments in a new and unique way insuring accurate patient identify and payment, patient safety, optimal health component(s) utilization, and usefulness.

2. An apparatus according to claim 1, in which said graphene chip cards encoded with said DNA and EEG potentiate signal capture and synchronization through physical proximity to a human brain via ear when using said SIM phone card method whereas electrical skin impedance as sensed by said FFT antenna bridges FFT Transform to blockchain meta-layer via RHIOTOKEN and derivative precious metal and diamond commodity backed RHIOCOINS over air gaps whereas said antennae captures impedance levels through heat and blood flow.

3. An apparatus according to claim 1, in which natural selection based emergent smart contracts comprised of neural network inspired software and said oracle hardware antennae evolve in real time within said web3 healthcare blockchain Mirror-Base utilizing new and novel healthcare cryptosystem blockchain through unique “API's” programming and oracles of said non-obvious emergent smart contracts as bridges to old, existing, and future—yet to be invented, blockchain projects.

4. An apparatus oracle according to claim 2, in which a PoP consensus protocol of said blockchain acts to validate records though proof of patient; proof of provider; and proof of pharmacy after proof of stake is performed using cryptocurrency rewards for participants of said commodity backed coins, or Central Bank Digital Currency backed, or Fiat backed in RHIOCOIN (RHC) of said blockchain.

5. An apparatus and system according to claim 4 in which real time payments and receipts in said multiple currencies and wallets for healthcare services are made to healthcare providers and linked to real time quality scoring matched to medical procedures, utilization, and results derived from statistical Data Envelopment Analysis with Best Case analytics based on mapping to desired subjective outcome parameters via the said healthcare cryptosystem native derived RHIOTOKEN and derivatives as acting units of Tokenization on the said blockchain smart contracts and deriving said RHIOCOINs as liquid representations of value representing provider fees, prices, claims, and insurer benefits that can be traded, gifted, received for said validation rewards, and purchased at any time for said payment to providers for services and other uses.

6. A distributed apparatus with systems and methods where according to claim 5 the said resultant token based smart contract coin of value backed by said physical commodities and said government backed cryptocurrency and physical fiat is secondarily limited against inflation by binding issuance and contract rules of promissory estoppel pegged to and consistent with the purchasing power of said units of currency at time of issuance and such that especially when used in said meta-verse-like healthcare cryptocurrency electronic layer where correcting of bad data is performed, and where said cryptocurrency tokens and coins remain liquid and where asset devaluation is averted avoiding currency pitfalls found in physical financial fiat systems without said promissory estoppel constraints that avoid systematic financial risk.

7. An apparatus according to claim 6 whereas linkage of said encoded smart contract graphene based oracle antenna chip and paired Web3 software coded Oracle antenna token act as one for synchronization and whereas bridge approach between said Web1 and Web2 applications to Web3 environments matches said bridge approach to and from physical to virtual assets and where said approach is transportable to additional industries beyond healthcare such as banking.

8. An apparatus according to claim 4 comprised of all US national and global international identifiable healthcare entity matching in HL7 protocol to FHIR protocol and USCDI found in certified EHR and/or open source electronic health products in the corresponding said blockchain medium additionally allowing patient access to every encounter in the longitudinal record in real time.

9. A dynamically generated standardized blockchain ledger formatted file according to claim 8 and representing a “Standard Designated Record Set” of a patient health record standard derived from an algorithm weighing HL7, US ONC Standards, FHIR, UM LS, EDI X.12, and USCDI.
